(19) United States
(12) Patent Application Publication
(10) Pub. No.: US 2012/0156177 A1
(43) Pub. Date: Jun. 21, 2012

(54) METHODS AND DEVICES FOR HARVESTING AND PROCESSING CONNECTIVE TISSUE PRECURSOR CELLS FROM AUTOLOGOUS FAT

(76) Inventor: MICHAEL A. SCARPONE, Bloomingdale, OH (US)

(21) Appl. No.: 13/269,556

(22) Filed: Oct. 7, 2011

Related U.S. Application Data
(60) Provisional application No. 61/391,075, filed on Oct. 7, 2010.
(60) Provisional application No. 61/427,210, filed on Dec. 27, 2010.

(51) Int. Cl. A61K 35/16; B01L 9/06; A61P 19/04; B04B 5/02

(52) U.S. Cl. 424/93.7; 494/16; 422/562

(57) ABSTRACT

Methods and devices are disclosed for repairing injured, infected, or otherwise damaged connective tissues. Such repairs use injectable combinations of: (i) stromal precursor cells, which can be obtained from a patient via liposuction, and which can mature rapidly into muscle, ligament, tendon, or other types of connective tissue cells that are needed at a repair site; and (ii) platelets, which will accelerate and promote a repair process, and which can be obtained from the patient's blood. When mixed together, stromal precursor cells and platelet cells will act synergistically, to generate new tissue which can replace or repair damaged tissue. Devices and methods are disclosed for processing an already-centrifuged layer of “spun fat”, obtained from a liposuction extract, to remove unhelpful materials and concentrate the stromal precursor cells, before the stromal cells are mixed with platelet cells for injection into a patient.

### RELATED APPLICATIONS

This application claims priority, under 35 USC 119, based on provisional application 61/391,075, filed on Oct. 7, 2010, and on provisional application 61/427,210, filed on Dec. 27, 2010.

### BACKGROUND

This invention is in the field of medicine and surgery. It relates to devices and methods for transplanting cells obtained from fatty tissue, in one region of a patient's body, into a different location in the same person's body, for the purpose of connective tissue repair.

When used to refer to surgical procedures, the terms “autologous transplantation” and “auto-transplantation” are used interchangeably. The term “autologous” indicates that living cells are removed, extracted, or otherwise obtained from one portion of a person's body, and are subsequently implanted, transplanted, injected, or otherwise emplaced in a different location in the body of that same person. Except in rare cases that do not require attention here (involving autoimmune disorders and the like), autologous cells (defined to include cells obtained from the same animal or human body that will be receiving the transplanted cells) do not create a risk of rejection by the patient's body.

In some situations, autologous transplanted tissues will remain in cohesive form. Examples include blood vessel grafts, skin grafts, etc. Those types of surgery are not relevant herein.

In the surgical procedures of interest herein, cells will be extracted in a liquefied form, from fatty tissue within a patient's body, using methods commonly referred to as liposuction. The term “lipo-” refers to fat or fatty; accordingly, “liposuction” is used broadly herein, to refer to and include any type of procedures which uses suction (via a hollow needle, cannula, or other tube) to remove fatty tissue from the body of a human or other mammal. Since suction of tissue through a needle, cannula, or other tube is involved, the tissue which passes through the tube will necessarily be in some type of a liquefied form, and “liquefied” is used broadly herein, to include thick and viscous cell suspensions that might also be referred to as a paste, slurry, sludge, or similar terms.

The phrase, “autologous fat grafting” (abbreviated as AFG) is often used in the medical literature, to refer to the types of procedures described herein. However, that phrase is not used herein, since it is potentially misleading, since “fat” will not be transplanted back into the patient. Instead, the desired and targeted cells that are useful for these types of procedures are a specialized class of cells, referred to herein interchangeably as either “stromal precursor cells” or “connective tissue precursor cells”. Those are described in some detail, below. Those cells are extracted from the liquefied fatty tissue that is obtained via liposuction, by means of processing described below, and the extracellular fat which initially accompanies those desired cells, in the liquefied tissue obtained via liposuction, preferably should be removed and discarded.

References herein to surgery (and to “surgical” procedures, operations, and the like) refer to and include medical interventions that involve physical manipulation of tissue (as distinct from, for example, diagnosing a condition and prescribing a drug to treat the condition). For the purposes of discussion herein, liposuction is deemed to be a type of surgery, and the medical interventions described herein are deemed to be “surgical” interventions. In the US and elsewhere, these types of procedures can be performed, lawfully, only by properly trained and licensed physicians; however, there are multiple thousands of physicians, in the US, who have the skills and ability to perform these procedures. Accordingly, even though substantial attention is devoted herein to the steps and devices that are used to carry out the procedures described herein, it should be recognized and understood that the level of “ordinary skill in the art” in this particular field includes physicians who already know how to perform liposuction, and who have done it multiple times, on multiple patients. Accordingly, the discussion herein of the steps, methods, devices, and equipment that are involved in liposuction must be regarded and understood as being mere summaries and overviews which are written for laymen, rather than as an instruction manual for physicians.

With regard to whether the term “surgery” is used appropriately to describe these procedures, it should be noted and understood that these procedures fall into a gray area, at the outer boundaries of “surgery”. A large number of medical procedures and interventions involve borderline areas, where it is not clear whether they fall within either classic or contemporary definitions of “surgery”. Indeed, an entire specialized branch of medicine has evolved during the past 20 years, which is commonly referred to as “sports medicine”. Most of the procedures that are performed by “sports medicine” specialists involve repairs to connective tissues, which includes muscles, tendons, and ligaments. Physicians who specialize in this branch of medicine frequently perform procedures that fall within the classical definition of “surgery” (i.e., they involve the physical manipulation and alteration of living tissues, which passes beyond merely handling fluids, such as withdrawing blood). As part of that work, specialists in “sports medicine” frequently use needles, cannulas, catheters, and other minimally-invasive tools, to manipulate tissue. However, they usually do not refer to themselves as “surgeons”, and they generally avoid the use of scalpels, incisions, or the types of surgery carried out by classical “surgeons” as that term is normally understood by laymen.

Accordingly, for the purposes of this invention, liposuction is deemed to be a form of “surgery”, since it involves the physical manipulation of tissue, and therefore falls within the classic definition of the term. However, it should be recognized that not everyone refers to it as “surgery”, and the “sports medicine” specialists who will carry out the procedures described herein usually do not refer to themselves as surgeons.

Returning to the subject of autologous transplantation of connective tissue cells, fatty tissue (also known as adipose tissue) is readily available in any human who is not exceptionally slender. It contains large numbers of cells which fall within a category referred to herein, interchangeably, as either “precursor” or “progenitor” cells. Both of those two terms indicate that these cells have reached a stage of partial differentiation, and maturation. At that stage of development, they are able to complete a maturation process which will convert them into any of several different types of fully differentiated “adult” cells.

Because these matters are crucial to understanding this invention, and because certain terms that are important herein have taken on various implications and subtleties that sometimes differ when used in the medical literature and the “popular press”, a digression is required to address some of the terms used herein.

### “Stem Cells” Versus “Precursor” or “Progenitor” Cells

Because of various factors that are involved in public, political, and legal battles over abortion and cloning, which are highly polarized and divisive areas, the term “stem cells” has taken on very different and in some respects conflicting meanings, depending on who is using the term, and how they are using it. Among reporters, the press, and the general public, “stem cells” implies and/or means the types of “embryonic” or “totipotent” cells which have a full, complete, and unlimited ability to develop into a complete adult. Accordingly, under the “general public and mass media” definition and interpretation, the term “stem cell” is used only to refer to cells which have the potential to differentiate into ANY type of cell that is found in a fully matured and differentiated adult.

However, in the scientific and medical literature, a very different set of meanings and implications arise. Under this definition, any cell that has not yet fully differentiated into an “irrevocably committed and fully differentiated” adult cell will properly fall within the term, “stem cell”. Stated in other words, any cell which still has a potential to differentiate into either of at least two (or more) different types of adult cells, is called a “stem cell”.

The medical definition arose, after developmental biologists discovered and realized that “stem cells” pass through a series of different stages, involving partial levels or degrees of what can be called “differentiation” or “commitment”. At the very earliest stage of embryonic development, immediately after a sperm cell and an egg cell have combined, the resulting cell (and then cells) are called “totipotent” or “omnipotent” stem cells, because they can become ANY type of cell found in an adult. However, that stage is very brief, and it lasts only until a fertilized egg has divided into about 4, 8, or 16 cells, depending on the species.

When stem cells pass beyond that early and brief “totipotent” or “omnipotent” stage (which lasts for only a few cell divisions), they become “multipotent” (also called “pluripotent”) stem cells. At that stage of maturation and “commitment”, they can still mature into numerous different types of cells, but the pathways they can follow begin to become constrained in various ways, and they can no longer form every cell type that exists in an adult.

For example, during the early development of a human or other mammalian embryo, some cells will move into a segment of developing tissue which will become the liver. For at least some period of time, these early progenitor cells will have the ability to become any type of liver cell, and there are numerous different types of liver cells. However, under natural conditions that exist within an embryo, an embryonic stem cell which has committed to becoming a liver cell will not be able to back up, move over to a different development pathway, and become a heart cell, a brain cell, or any other type of non-liver cell.

Throughout all stages of infancy, adolescence, and adulthood, every internal organ contains numerous “multipotent” or “pluripotent” stem cells. In any particular organ, these types of cells will retain the ability to complete a process of differentiation and maturation, in a manner which can create a variety of different types of new cells, which can replace aging cells that can no longer fully perform their cellular functions. This is an absolutely crucial function, which is described in more detail below, and it helps explain the surprisingly large number of such cells, in fatty tissue. Under the medical definition, these types of “multipotent” or “pluripotent” precursor cells are labeled as “stem cells”. However, that use of the term directly conflicts with the “general public and mass media” definition of “stem cells”.

Accordingly, to avoid “unwanted baggage, and unintended meanings” that can arise when “stem cells” are being discussed, that term is not preferred herein, and terms that are more scientific and less divisive are used, such as “progenitor” or “precursor” cells.

Two additional terms requires attention; those terms are “stroma” and “stromal”. In medical terminology, the term “stroma” refers to the biomechanical framework (or scaffolding, matrix, support, or similar terms) of an internal organ, muscle, or similar non-bony tissue. The corresponding adjective is “stromal”. Accordingly, stromal cells include cells which contribute to the strength, cohesiveness, flexibility, elasticity, integrity, and other structural and biomechanical traits of an organ, muscle, tendon, ligament, or other type of connective tissue. Since the types of stromal tissue that is of interest herein is also referred to as “connective tissue”, the term “stromal precursor cells” is used interchangeably with “connective tissue precursor cells”.

Briefly, “stromal precursor cells” includes cells which still have an ability to differentiate and mature into any of several different types of connective tissues, which can include muscles, tendons, ligaments, etc. Accordingly, these types of precursor cells are of great interest to “sports medicine” specialists, and to any physicians who are faced with the task of repairing, reconstructing, or supplementing various types of connective tissues that have become injured, infected, chronically sore, or otherwise damaged, or which suffer from congenital defects, problems that have arisen due to aging or senescence, etc. For convenience, connective tissue which is suffering from any of those types of conditions, at a level severe enough to require or merit medical intervention as described herein, is referred to herein as “damaged” tissue.

### Types and Examples of Connective Tissue Repairs

Because of various aspects of human physiology and activity (which lead to different types of stresses being imposed on different joints), the most common types of connective tissue injuries or problems involve certain specific joints, including the following:

1. rotator cuff problems, problems involving certain other muscles, tendons, and/or ligaments, in shoulder joints;

2. hip and/or groin problems, involving either “hyaline” cartilage (i.e., the type of smooth-surfaced cartilage which coats a bone surface in an articulating joint), or various types of strains, tears, or other injuries to muscles, tendons, or ligaments in the region of the pelvis and upper thighs;

3. knee problems, which can include cartilage, ligament, tendon, or muscle problems;

4 ankle problems;

5. finger, thumb, wrist, or hand problems; and,

6. elbow problems, often referred to as “tennis elbow” regardless of whether tennis was involved.

In addition to those types of problems, various types of skin ulcerations (typified by bedsores, also called decubitis ulcers) occur in various types of patients, especially patients suffering from diabetes, obesity, or other types of circulatory or metabolic problems. These types of ulcers, which typically are called skin ulcers to distinguish them from stomach ulcers (and to identify them as a class of ulcers that are readily visible on skin surfaces), frequently involve damage to underlying tissues as well, including muscles, tendons, and ligaments. They occur most frequently around the feet and ankles (because of circulatory issues), or on body surfaces that tend to be pressed against bedding materials for hours at a time during sleep, especially among the elderly.

In addition to those factors, stromal precursor cells can also be useful for reconstructing or altering the appearance of various types of scars, and for similar types of surgery which can be generally classified as cosmetic surgery (i.e., surgery, injections, or other interventions in which alterations to appearance are of primary or major importance).

Accordingly, the types of “stromal precursor cells” which have already reached a state of partial differentiation, and which can complete a maturation process that will convert them into muscle, tendon, ligament, or other connective tissue cells, are of great interest, for the types of medical treatments described herein. Stromal precursor cells from autologous fatty tissue can be used to help repair muscles, tendons, ligaments, and other connective tissues that have been injured or otherwise damaged in various ways, or which have been surgically removed or otherwise manipulated (such as during removal of a tumor, cyst, or other injured, damaged, or unwanted tissue). They also can be used to help repair, regenerate, or replace tissue that was damaged by infection, trauma, disease, or other events, conditions, or problems. Accordingly, if properly performed, these types of autologous transplantations of stromal precursor cells, from fatty tissue, can help patients recover from sports injuries, injuries suffered during various types of accidents or trauma, infections by pathogenic microbes that attack connective tissues, and similar problems. They also can be used for various types of cosmetic and/or reconstructive surgery, such as for scar revision, repair of cleft palates, etc.

Importantly, those types of stromal precursor cells are present in surprisingly high numbers, in fatty tissues, because of an important cellular and physiological process.

### Apoptosis; Active And Ongoing Cell Replacement In Soft Tissues

As mentioned above, because of a crucially important and highly active cellular process that occurs in all mammals, there are surprisingly large numbers of stromal precursor cells, in fatty tissue. While it is not crucial to fully understand the natural cellular process in order to understand this invention, a working knowledge of it can help the reader reach a better understanding of how and why this invention works.

Cells that reach an advanced stage of aging are often referred to as “senescent”, which derives from the same root word as “senile”. In the same way that a senile person might be able to live for many years, if properly taken care of by other people, senescent cells remain viable, as living cells, but they have lost the ability to fully perform their normal functions.

Severe health problems and major biological inefficiencies would arise, if muscles or other tissues in an animal had to devote part of their resources to nurturing, nursing, and providing for senescent cells, in an effort to keep them alive for as long as possible even after they lost the ability to perform their essential functions. To avoid those problems, mammalian cell biology evolved in a very different direction. Rather than keeping cells alive for as long as possible, even after they can no longer perform their essential functions, mammalian biology evolved in ways that rapidly identify, kill, recycle, and replace aging and senescent cells with new cells, in a constant process of cell turnover and replacement. This process involves a well-known cellular activity called “apoptosis”, sometimes referred to as “programmed cell death”. This process is controlled by mitochondria, which are tiny “organelles” that have their own membranes, inside mammalian cells. These organelles are the remnants of tiny anaerobic bacteria, which initially invaded larger cells, and which then created a symbiotic relationship with their “host” cells. A typical cell has dozens or even hundreds of mitochondria, inherited entirely from the mother, with no genetic contribution from the father. These organelles are enclosed within their own membranes, and they carry their own DNA, which even has its own specialized genetic code, which is different from the genetic code used by the chromosomes in the nucleus of a cell.

Mitochondria are the “cellular furnaces”, where glucose (the main energy supply for any mammalian cell) is oxidized and “burned”, to convert the glucose into carbon dioxide and water, in a way that releases energy which a cell can use. As a result, the chemical mixtures inside mitochondria are relatively harsh, especially since large numbers of destructive oxygen radicals are constantly being generated, as a byproduct of glucose oxidation in the mitochondria. As a result, the membranes which enclose the mitochondria are chemically attacked and degraded, at relatively rapid rates.

In an aging cell, when the mitochondrial membranes become worn out and degraded to a point where they become permeable, they begin releasing a specific molecule called “cytochrome c”. That messenger molecule activates a sequence of events, which will culminate in a “macrophage” (a specialized white blood cell) engulfing, killing, and digesting the aging cell. This frees and releases molecular building blocks (such as amino acids, nucleotides, etc.), which will be used to make new cells, to replace the senescent cells that were killed and digested.

That type of “programmed cell death” is essential for keeping muscles, tendons, ligaments, internal organs, and other tissues vigorous and fully functional for spans of time measured in years or even decades. Except for neurons (which are in a special class), the typical lifespan of any particular cell, in any large animal, is only a few weeks or months, and the process which recycles and replaces aging cells with new cells, in any particular type of tissue, is very active at all times.

Therefore, the presence of large numbers of precursor cells which have reached a moderately advanced stage of development and differentiation, and which can complete the final steps of maturation into any particular type of “adult” cell which is needed in some particular location at a specific time, is crucially important to the process of constantly replacing aging cells with new cells. In any organ, joint, or other “subassembly” which contains multiple cell types, a rich supply of partially-differentiated precursor cells, which required a substantial amount of time to reach that stage of development but which can rapidly undergo the last and final steps in a differentiation and maturation process that will create fully “adult” cells, is an essential part of the natural process of replacing old cells with new cells.

That is a well-known feature of mammalian physiology, and over the past decade, major advances have been made that allow physicians to extract and obtain large numbers of stromal precursor cells, from fatty tissues that can be obtained in a minimally-invasive manner, via liposuction.

### Conventional Liposuction Procedures and Equipment

Conventional and well-known methods can be used to obtain stromal precursor cells from fatty tissue, via liposuction. These methods are taught in courses that are taken by surgeons and other doctors who wish to learn to perform these procedures, and the types of cannulae, syringes, and other kits, devices, and machines that are used during this type of liposuction are readily available. For example, a website at www.viafill.com illustrates the VIAFILL™ system, sold by the Lipose Corporation and specifically designed for the type of liposuction described herein.

Rather than resembling the types of enlarged cannulae that are used to remove large amounts of fatty tissue for the purpose of weight reduction, a cannula designed to harvest viable stromal cells for autologous transplantation has dimensions that are similar to an enlarged hypodermic needle. This type of cannula is made of a rigid metal alloy, and has a smooth rounded tip that will not readily puncture, cut, or damage muscles or membranes. A series of medium-sized holes (or orifices, channels, or similar terms) pass through the sides of the barrel, in a region near the tip of the cannula.

Before this type of cannula is inserted into a patient's body, a hypodermic needle (with a very thin barrel, and a very sharp beveled tip) is used to inject a topical anesthetic (such as xylocaine) under the skin, at the location that will be worked on. Typically, the first anesthetic needle is withdrawn, and after the first batch of anesthetic drug has taken effect and partially numbed the area, a second hypodermic needle is inserted, and moved around a semi-circular area, to spread additional anesthesia into the area beneath the skin (this is often called a “fanning” procedure or pattern, since the affected area resembles the shape of a semi-circular hand-held fan). The needle is kept nearly tangential to the skin, so that the tip does not penetrate into any major muscles, and remains in a shallow layer of fat between the muscles and the skin.

Typically, as the second anesthetic needle is being withdrawn, the sharp tip is used to create an enlarged nick in the skin. The smooth rounded tip of an injection cannula (which closely resembles or which can be identical to the extraction cannula) is inserted through that nick in the skin, into the fatty layer. That cannula is used to inject a buffered saline or similar aqueous solution into the fat, to help liquefy the fat. This increases the quantity of fat (and the number of viable stromal cells) that can be extracted from a relatively small region beneath the skin. The aqueous liquid emerges from holes in the side of the cannula, near the tip, and by using a combination of liquid injection and cannular motion, the physician can create, in a relatively brief time, a region of liquefied fatty tissue that is ready for extraction.

When that point is reached, the injection cannula is withdrawn, and an extraction cannula is inserted in its place. To minimize any risk of unwanted complications or damage to tissue in the surrounding region, most surgeons operate an extraction cannula solely by using their hands to exert tension on the plunger handle, within an extraction syringe, without using a pump or other machine to create an artificial suction. By closely watching the flow of viscous fluid into the clear-walled barrel of a syringe while sustaining a continuous and reliable “feel” (through their hands) for what the cannula is doing beneath the skin, a skilled surgeon can develop a reliable sense of how to extract a substantial volume of fluid in the safest possible manner, with minimal scarring, tissue disruption, or unwanted alterations or deformation of the skin surface contour in the affected region.

The only mechanical device which a surgeon typically uses, to help sustain a continuous and relatively stable level of suction force on the extraction cannula, is a clip-type device commonly called a “Johnnie-Lok”. By means of a simple twisting motion, this type of clip (which rests and presses directly against the syringe opening) can be used to temporarily secure the plunger handle to the syringe barrel, at any position along the length of the plunger handle. Accordingly, a surgeon can pull out the plunger handle until a desired level of suction force is reached, and then use the “Johnnie-Lok” clip to sustain that level of suction, for some period of time, while the surgeon focuses on the movement, positioning, and “feel” of the cannula tip beneath the skin. When enough fluid has entered the syringe barrel to cause the suction force to drop to an undesirably low level, the surgeon twists the plunger handle to release it from the Johnnie-Lok, pulls the plunger farther out of the syringe barrel to re-establish a desired and effective level of suction, and then clips the plunger handle to the syringe at the new position.

This suction process continues until the syringe is nearly filled with liquid. At that point, the full (or loaded, etc.) syringe is removed, and a new and empty syringe is put in its place, to extract more fluid. This replacement can be done in either of two ways: (i) by detaching a full syringe from the extraction cannula while leaving the cannula in place, in the patient's body; or, (ii) by pulling out the cannula, and replacing it (this is often done is a surgeon suspects one or more of the holes in the cannula have become clogged).

As many syringes are used as are necessary to remove a quantity of liquefied fatty tissue that the surgeon believes to be useful and desired for a particular procedure on a particular patient. These volumes are important, and they are discussed in more detail below, because they factor heavily into the specific teachings and claims of this invention.

Returning to background information which can help explain how this invention works, fatty tissue is a complex mixture which consists mainly of four types of material:

(1) collagen, an extra-cellular fibrous protein which forms a three-dimensional “matrix” or “scaffold” which holds the cells together in essentially all connective tissues;

(2) large numbers of living cells, most of which will be attached or “anchored” to the extra-cellular collagen matrix;

(3) aqueous fluid, which comes from two main sources: (i) the saline solution or other artificial fluid that was injected into the extraction site, to help liquefy the fatty tissue; and, (ii) the naturally-occurring aqueous fluids that are present even in fatty tissue, mainly in the form of lymph and “tissue gel”, both of which help nutrients reach and permeate into cells, and help carbon dioxide and other wastes diffuse away from the cells; and,

(4) a compound called “glycogen”, which is a polymerized fatty compound used by mammals for energy and food storage. As described in any textbook on physiology, glycogen is created by “stringing together” molecules of glucose (a specific 6-carbon sugar, which can be readily and rapidly metabolized by all mammalian cells). When additional energy supplies are needed, the body can begin cleaving off individual glucose molecules, one at a time, from strands of glycogen. Each molecule of glucose can then be metabolized by the cellular process called “glycolysis”, in which glucose molecules are “burned” as a fuel source, in a manner which oxidizes the glucose into carbon dioxide and water, and which releases energy during the oxidation process.

Accordingly, when fatty tissue is converted into liquefied form (with the aid of injected saline solution or similar liquid), for extraction by a cannula, collagen fibers must be broken, and a relatively thick and viscous mixture is removed, which contains still-living and viable cells.

There are two main types of uses for that type of fatty tissue, after it has been extracted. One is for breast augmentation, in which careful placement of volume and bulk is the most important factor for achieving a desired cosmetic effect. The cell-concentrating procedures and implantation methods described herein can be adapted for such use, if desired (especially for use in reconstructive surgery after a lumpectomy or breast removal, in a patient suffering from breast cancer, which does indeed involve “connective tissue repair” as that term is used herein). Either of those types of surgery (i.e., breast enlargement for purely cosmetic purposes, or breast reconstruction following tumor removal) are specialized types of surgery; they are well-known to cosmetic surgeons, and they can be carried out by surgeons who specialize in that type of surgery, using methods and cell preparations as disclosed herein, if desired. However, that type of cosmetic surgery is not of primary interest herein.

Although that type of cosmetic surgery can adapt and utilize the procedures and equipment described herein, the primary and central focus of these types of treatments will involve connective tissue repairs, as listed and summarized above. Accordingly, the discussion herein focuses on those types of uses.

Before the details of the invention herein are disclosed, one other type of concentrated cell preparation, which was developed years ago and which is well-known and widely used, merits attention.

### Combining Stromal Precursor Cells with “Platelet Rich Plasma”

It was discovered, at least 10 years ago (e.g., Abuzebi et al 2001), that the use of stromal precursor cells, obtained from fatty tissues, will generate better results in connective tissue repairs, if the stromal cells are mixed or otherwise coadministered simultaneously with a blood-derived preparation called “platelet rich plasma” (PRP). Machinery and equipment (including “kits” with all necessary disposable devices and supplies) for creating PRP, from a patient's blood, are readily available to physicians, in terms of relatively small and convenient “desktop” devices that are roughly the size of a typical microwave oven used in a home. Accordingly, this aspect of the invention is old and known, and this section merely provides an overview and background information on platelet-rich preparations from blood.

Platelets are specialized blood cells, which can be obtained from circulating blood. In mammals, they are heavily involved in the natural healing processes which arise in response to any type of injury. Accordingly, they were recognized by at least the 1980's as a rich and available source of: (i) growth factors, including hormones that will trigger cell reproduction; (ii) proteins that can recognize and bind to the “torn ends” of collagen fibers, at the site or a wound or other injury; and, (iii) other biomolecules which can help promote and accelerate healing.

Briefly, when “whole blood” is processed (often called “fractionated”) in a centrifuge, at an appropriate speed which will not damage or kill blood cells (often expressed as a multiple of “gravity” force, such as 40 g, which is commonly used in blood fractionation), three main layers will form.

The “bottom” layer will contain red blood cells (RBC's, also known as erythrocytes), since RBC's have the highest weight-per-volume density of any of the blood components. Red blood cells do not contain any chromosomes; they are formed by a simple and rapid process of cell “budding”, rather than cell replication, and they last for only a few days after they are created. Since their sole functions are to deliver oxygen and remove wastes, which require active circulation of blood, they would only clutter and clog up an injured area which has been packed with a stationary stromal-and-platelet cell mixture, for connective tissue repair as described herein. Therefore, the red blood cells are discarded, when a PRP mixture is being created for use in wound healing.

Above the bottom layer of red blood cells, a center layer will form, which mainly contains platelet cells, and white blood cells (leukocytes).

The uppermost layer, which is relatively clear but with a yellowish tint, contains the carrier liquid, which is blood plasma. If blood plasma is further processed to remove fibrinogen and other clotting factors (which are extra-cellular proteins), the resulting liquid is called blood serum.

In a broad sense, any blood preparation in which the platelet concentration has been enriched above a “baseline” level (as found in unprocessed “whole blood”) can be referred to as “platelet rich plasma” (PRP). In order to render PRP truly effective for accelerating and enhancing wound-healing activity, the concentration of platelets preferably should be increased to at least about 5 times (5×) their “baseline” level.

After an initial centrifugation step has been used to remove red blood cells from a processed blood fraction, other processing steps (including various types of filtration, gel processing, “affinity binding”, or chromatographic steps, and possibly involving the addition of various other agents) can be used, if desired. Depending on the specific steps that are used, this type of additional processing can, for example: (i) remove other types of white blood cells from a concentrated platelet preparation; (ii) remove one or more specific targeted proteins (such as growth factors, albumin, etc.) or other molecules; (iii) remove a portion or fraction of the plasma liquid, from the platelets, to further increase platelet density and concentration levels; or (iv) add various supplemental agents to a PRP preparation.

These types of processing steps, which can be used to create PRP mixtures for use in wound-healing procedures, are described in numerous review articles, including Pietrzak et al 2005, Maniscalco et al 2008, Hall et al 2009, Gociman et al 2009, Lacci et al 2010, and Lopez-Vidriero et al 2010.

Various companies sell processing machines that are specifically designed to extract PRP, from whole blood. A preferred system which has been used with good results by the Applicant herein is the SmartPReP™ Platelet Concentrate System, sold by Harvest Technologies. It is illustrated and described at www.harvesttech.com and in additional sources which can be identified and obtained from that website. That system generates a PRP extract with high numbers of platelets and monocytes, which are beneficial for the types of treatments described herein, and with reduced numbers of other white blood cell types (including granulocytes, which are not especially beneficial for connective tissue repairs as disclosed herein).

Other companies also make machines which can generate PRP extracts from whole blood; one example is the MAGELLAN™ system, sold by the Arteriocyte Company (http://arteriocyte.com).

Both types of machines were introduced into the marketplace in the U.S. in 2009, and those two companies are competing vigorously against each other to create superior machines. Accordingly, enhancements in both machines are likely to occur in the coming decade, which will further optimize PRP extraction methodology.

Accordingly, methods which utilize mixtures and combinations of: (1) stromal precursor cells, obtained from fatty tissue via liposuction; and, (2) platelet cells, obtained and concentrated from blood samples, have been known and in use for roughly 10 years, for connective tissue repairs. These methods have established an important specialty, within the field of medicine and surgery, and this current invention involves improved methods and devices which can enhance and improve those types of surgical and medical interventions and procedures.

Accordingly, one object of this invention is to disclose and provide methods and devices that can help achieve greater efficiency and better results, when stromal (connective tissue) precursor cells are obtained from fatty tissue, via liposuction, for autologous transplantation into the same patient, for connective tissue repairs.

Another object of this invention is to disclose and provide certain types of specialized devices, which include both reusable and disposable components that will interact with each other, which will enable improved methods of centrifugation and other processing steps, for handling and concentrating stromal precursor cells which will be used for autologous transplantation.

Another object of this invention is to disclose methods and devices for carrying out a process of concentrating and enriching autologous cells that have been harvested from fatty tissue extracted from a patient via liposuction, with essentially no loss of stromal precursor cells from the fatty tissue extract.

These and other objects of the invention will become more apparent from the following summary, drawings, and detailed description.

### SUMMARY OF THE INVENTION

Methods and devices are disclosed for processing stromal precursor cells (i.e., cells which can differentiate into various types of connective tissue cells, such as cells in muscles, ligaments, tendons, etc.), which can be obtained in large nubers from fatty tissue extracts, obtained via liposuction. When mixed with a second cellular mixture called “platelet-rich plasma” (PRP), which can be obtained by processing blood from the same patient, the stromal precursor cells can be used for autologous cell transplantations, to repair, regenerate, or supplement connective tissue, at an injury site or other location that is in need of repair or other medical intervention. The processing of the fatty tissue extract involves a combination of centrifugation and filtration, during an initial separative treatment designed to concentrate stromal precursor cells from the liposuction fluid. The layer that will be isolated by centrifugation can be referred to by terms such as “spun fat”.

Preferably, the “spun fat” should be treated by a second stage of processing as well, to further concentrate the stromal precursor cells while eliminating glycogen and fat. In one embodiment, the cell suspension can be incubated with collagenase, an enzyme, to break down and remove the extra-cellular collagen fibers that will be present in the initial fatty extract. In an alternate embodiment, which is preferred because it will not require extensive clinical trials to obtain FDA approval, mechanical means (such as passage of the “spun fat” cell suspension through an extruding device, vibrating screen, or high-shear stirring vessel) can be used to detach stromal precursor cells from the extra-cellular collagen matrix. A second centrifugation step, or a filtration step, can be used to further concentrate the stromal precursor cells that have been released form the fatty matrix of the spun fat material.

By means of these processing steps, the stromal precursor cells from a fatty liposuction extract can be converted into a highly concentrated form, for subsequent reintroduction (along with platelet-rich plasma, if desired) into a wound site or similar area that is in need of tissue repair.

Among other features, the invention herein discloses specialized centrifugation cartridges, which will enable up to 120 cubic centimeters (cc) of fluidized liposuction extract, in six syringes which will hold 20 cc each, to be processed in a single centrifugation step, using the same centrifuge machine that is also used to prepare platelet-rich plasma (PRP). To accomplish that, each 20 cc syringe must be short enough to fit into an accommodating centrifugation cartridge which will contain three wells, to hold up to three syringes at a time. These types of centrifuges hold two cartridges at a time, at opposite ends of a rotor, for proper balance during high speed centrifugation). Accordingly, a set of two cartridges, holding three syringes each, will hold six syringes, containing up to 120 cc of liposuction extract with stromal precursor cells. It has been found that 120 cc of fatty tissue extract is sufficient to satisfactorily handle the vast majority of such procedures, even in cases where a patient will require a series of multiple cell injection procedures over a span of multiple months.

Accordingly, improved methods are disclosed herein, for multi-step but convenient preparation of highly purified stromal precursor cells, for use in connective tissue repairs. In addition, an improved combination of centrifuge cartridges and liposuction syringes are disclosed herein, to provide a single set of devices that will enable convenient and efficient handling of up to 120 cc of liposuction extract at a time, using the same centrifuge machine which is used to prepare platelet-rich plasma.

### BRIEF DESCRIPTION OF THE DRAWINGS

FIG. 1 is a schematic depiction of the layers that will be formed, when a liquefied fatty tissue extract, obtained from a patient via liposuction, is centrifuged for a suitable period of time at a speed that will not damage viable cells. This drawing shows two identical syringes, each containing approximately 20 milliliters (mL or ml) of a fluidized liposuction extract which has been centrifuged; both syringes are held within accommodating wells, in a cartridge designed for use in a centrifuge machine. The uppermost oily layer (with the lowest density) will be discarded. The center layer, referred to herein as “spun fat” for convenience (and as “concentrated fatty tissue extract” in the claims) will contain a semi-concentrated suspension of stromal precursor cells (which can also be called connective tissue precursor or progenitor cells). The bottom layer, which has the greatest weight-per-volume density, will contain mostly water, but it will also contain substantial numbers of stromal precursor cells; therefore, that watery layer will be forced, using gentle pressure and/or suction, through a filter which does not allow cells to pass through. This will retain the stromal precursor cells on the surface of the filter; they can then be washed off of the filter surface, and mixed with cells in the “spun fat” layer, for additional processing.

For comparative and descriptive purposes, one of the syringes in FIG. 1 is shown in a well that has a valve-controlled flow conduit which leads to a filtering device. The other syringe is shown with a simple “cap” screwed to its tip. The first arrangement is feasible, but for reasons described below, the second arrangement is believed to be preferable.

FIG. 2 is a flow chart depicting a series of steps that can be used to process, extract, and utilize stromal precursor cells contained in a liposuction extract from a patient who requires connective tissue repair.

FIG. 3 is a schematic depiction of a device for mechanically separating stromal precursor cells from fat and collagen fibers, in a “spun fat” suspension created by: (i) initial centrifugation of a liposuction extract, followed by (ii) mixing the spun fat material with a saline buffer containing a gently surfactant, such as lecithin. A piston or plunger is used to force the cell suspension downward, through an extrusion plate having multiple small tapered orifices passing through it. This will partially break apart the spun fat layer, in a manner that will begin releasing the stromal precursor cells from the fat and collagen matrix. The mixture of water, fat, and cells is circulated and rotated, with the aid of a mechanical stirring device, around a cylindrical chamber having several perforated “catch plates” (for simplicity, only one such catch plate is shown in FIG. 3). Fatty and oily droplets in the aqueous suspension will impinge against and cling to the catch plates, while the stromal precursor cells will drop out of solution and accumulate on a sloped floor of the cylinder, for removal via an outlet.

FIG. 4 is a schematic depiction of a “screen passaging” system which can be used to mechanically separate viable cells from the fat and collagen matrix in a “spun fat” suspension created by centrifuging a liposuction extract.

FIG. 5 is a schematic depiction of a “shearing-force stirring” system which can be used to mechanically separate viable cells from the fat and collagen matrix in a “spun fat” suspension created by centrifuging a liposuction extract.

FIG. 6 is a perspective view which depicts a centrifugation cartridge that can hold three syringes, with each syringe holding up to 20 cc in volume. This centrifugation cartridge is sized and designed to fit into a commercially available centrifuge machine that is designed and suited for preparing platelet-rich plasma (PRP) from whole blood.

FIG. 7 is an elevation (plan) view of two centrifugation cartridges, each having three wells for syringes that can contains 20 cc each of liposuction fluid. Each cartridge is held within a basket device at one end of a rotor, in a centrifuge. The wells in the cartridge are labeled, to indicate a loading sequence that will maintain balance and symmetry regardless of whether two, four, or six syringes are loaded into the centrifuge. A small additional well is also included in each cartridge, which can hold water or any other liquid or weight, to evenly balance the weights of two cartridges (including any loaded syringes held by the cartridges) against each other.

### DETAILED DESCRIPTION

As summarized above, this invention relates to improved devices for processing stromal precursor cells (i.e., cells which can mature into connective tissues, such as muscles, tendons, and ligaments). Those types of cells can be obtained from fatty tissues in a patient's body, by means of liposuction. If processed properly and then mixed with platelet-rich plasma (PRP), those types of precursor cells can be extremely useful and helpful in treatments for various types of tissue defects, including but not limited to treatments for injuries, infections, arthritic or similar degeneration, scars, or other problems that are associated with joints, limbs, or other connective tissues.

Conventional liposuction procedures, which have been used with good results by the Applicant herein to obtain supplies of stromal precursor cells from patients, are described in Example 1. However, this invention does not depend upon, arise from, or claim any specific procedures or details relating to any process or method used to extract fatty tissue from a patient. When human patients are involved, liposuction (or any other form of adipose tissue extraction) is a form of surgery, and can be performed only by qualified and licensed physicians. Any physician who performs liposuction will have his or her own preferences concerning equipment, methods, and kits with disposable supplies, for performing liposuction.

Various devices and supplies for performing low-volume liposuction (i.e., the type of liposuction procedures that are suited for harvesting stromal precursor cells, as distinct from high-volume liposuction, as used for weight reduction purposes) have been available for years, and are well known to those skilled in this field of surgery. As one example, the VIAFILL™ system, sold by the Lipose Corporation, includes various tools and instruments (generally referred to as “devices” herein, to distinguish them from liquid-type supplies, reagents, pharmaceuticals, etc.) that are specifically designed for low-volume liposuction. Those are described and illustrated at www.viafill.com.

However, there is one factor that requires attention, with regard to the liposuction part of the treatments described herein. This factor relates to the volumes of fatty tissue that should be extracted, for carrying out various different types of treatments.

### Liposuction Extraction Volumes

Based on trials and tests on a number of patients (all of whom provided informed consent), which involved treatments for some of the most common types of connective tissue injuries that can benefit from stromal precursor cell transplantations as described herein, the Applicant herein has determined that volumes such as those listed in Table 1 usually are appropriate in most cases of typical pain, discomfort, and damage, when treating an adult male. These volumes will include aqueous fluids that were injected into a patient to help break apart and liquefy the fatty tissue that was being removed, and which was then suctioned out along with the fatty tissue. Accordingly, volumes of actual fatty tissue which are extracted during these procedures, when injected aqueous fluids are not included, are correspondingly lower. Furthermore, when treating cases of severe damage and pain, correspondingly larger volumes should generally be extracted, in anticipation that a series of several injections will be required, over a span of months, to provide better results.

### Table 1

### Typical Fluid Extraction and Processed Cell Volumes

xxx

[symbol]

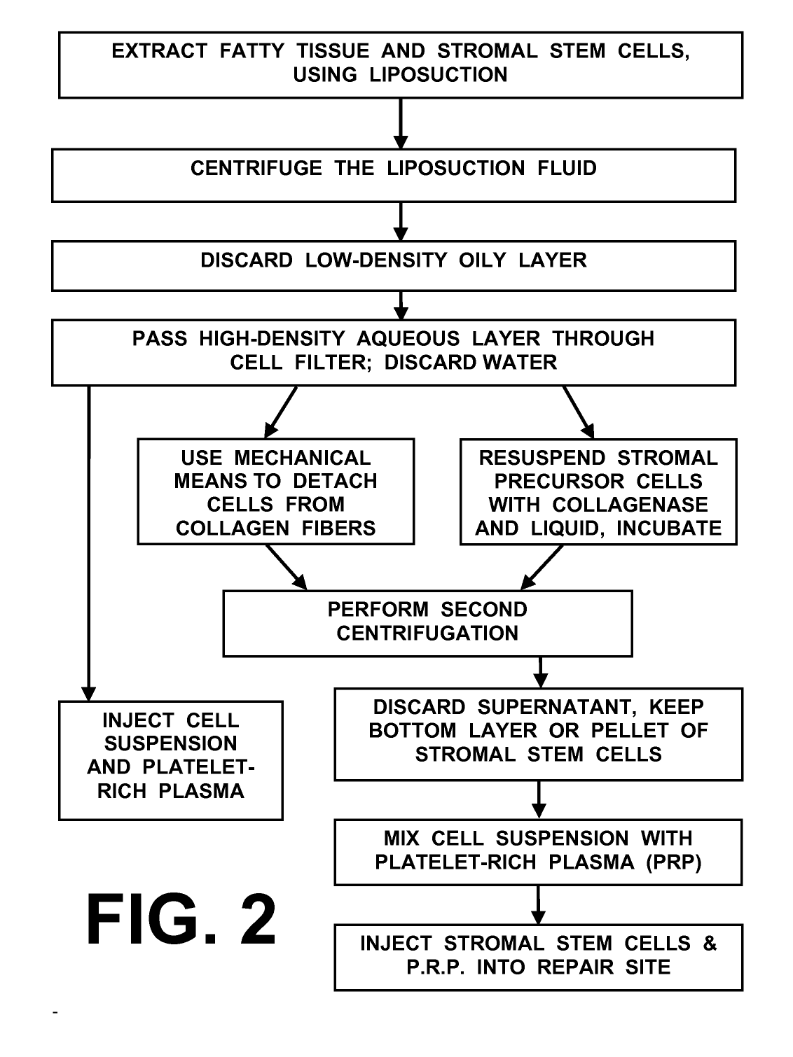

Table 1 also indicates the approximate volumes of fully-processed stromal precursor cells which should be obtained via these types of liposuction procedures, when the cells have been fully processed and concentrated, prior to injection back into a patient. These concentrated cell volumes will be contained in a cellular pellet or layer, which will be generated as described below and as indicated in FIG. 2.

If it appears that inadequate numbers of cells are present in the fatty tissue extract from some particular patient, then the treating physician can extract additional fatty tissue, either from the same area that has already been anesthetized, or from a different area of the patient's body (such as (i) if the anesthesia has partially worn off, and/or (ii) if the withdrawal of additional fatty tissue from the initial liposuction site might leave undesirable surface irregularities). If a physician wishes to do so, he or she can check the apparent cell density per volume of fluid, in one or more small samples of fluid taken from the “spun fat” material after the first centrifugation, or from a sample of liquid taken at any stage during subsequent processing. That type of check, for cells-per-volume density, can be performed using a light microscope if desired, or by means of more sophisticated equipment, such as a flow cytometer.

An important factor which must be taken into account, when planning and performing liposuction to obtain a sufficient quantity of stromal precursor cells for any particular treatment, arises from the fact that certain types of injuries can require two or more treatments, at different times, to obtain optimal results. Such repeated treatments normally will involve: (i) a first injection of stromal precursor cells mixed with PRP, usually on the same day that the liposuction is performed; and, (ii) one or more additional treatments, which typically will occur at least a month or more after the initial treatment.

For example, if a patient requires “bilateral” treatments (i.e., on both sides of the body, such as on both hips, or both knees), a preferred approach usually will involve: (1) treating a first hip or knee, which usually will involve the joint that is causing the most pain or discomfort at that time; and (2) giving the patient 2 or 3 months for that treatment to take full effect, while the patient undergoes a training, rehab, strengthening, or similar program which will focus on the treated joint, without any interference or impairment that would occur if both joints on both sides were treated in a single session. Subsequently, after the initial treatment on one side has fully settled in and stabilized, the second joint will be treated, and that second treatment will require its own rehab, strengthening, or similar program following the treatment.

Other situations also arise in which stromal cell treatments will provide greater benefits if repeated one or more times. For example, a number of patients who suffered from various types of problems (include the types of arthritis that involve damage to cartilage, in joints such as knees or hips) benefitted from a series of two or more treatments as disclosed herein, at the same treatment site. Patients in this category commonly reported that an initial treatment provided a large measure of relief, such as by eliminating 60 to 90 percent of the initial pain. Those patients chose to undergo a second treatment, to determine whether the second treatment could provide even more relief. Accordingly, followup treatments for patients in this class can be regarded as comparable to “booster” shots. When these types of repeated treatments are involved, the entire series of treatments, taken together, will require a larger volume of liposuction extract.

It also should be noted that pellets or layers of processed and compacted stromal cells, prepared as described herein, have been stored for up to six months in medical-grade freezers (which can sustain temperatures such as −80° C., which is in the temperature range of liquid nitrogen), with no signs of cell damage or loss of viability.

Accordingly, based on those initial tests that have been performed to date, the Applicant herein has discovered that if liposuction is used to extract liquefied fatty tissue in volumes of up to 120 cc (which will include a significant quantity of injected saline solution, mixed with the fatty tissue), then that 120 cc volume will enable the treating physician to meet the needs of the very large majority (such as more than about 95%) of all patients who are in need of these types of treatments, including patients who need a series of repeated treatments.

Therefore, this invention discloses a set of centrifuge cartridges which can hold and accommodate six syringes at a time, with each syringe holding 20 cc of fluid, and with all of the syringes sized to fit into a centrifuge cartridge that will fit into a “desktop centrifuge” machine.

As indicated by the name, a “desktop centrifuge” has dimensions that enable it to sit, in a stable manner at all times during loading, operation, and unloading, on top of a desk, table, laboratory-type bench, or similar furniture-type surface with a conventional height (such as from roughly mid-thigh, to slightly above waist-high, on an average adult), while providing full visual and working access to a lid or other movable top compartment which is low enough to allow an average adult of normal height to lean over, look into, and load and unload the machine while standing normally on the floor next to the desk, bench, etc.

Desktop centrifuges are well-suited for use in physicians' offices, and they are notably smaller than a substantially larger class of “free-standing” centrifuge machines which are designed for full-time laboratories. Most “free-standing” centrifuges are comparable in size to a washing machine; they are designed to enable fluid-holding vessels that are much larger than 20 cc syringes to be loaded into such machines, and if a “free-standing” centrifuge were sitting on a typical desk or laboratory benchtop, a typical adult would not be able to lean over it and look into it. Numerous types of desktop centrifuges have been commercially available for decades, and they are in widespread use in the offices and clinics of large numbers of physicians.

There is no need for a centrifuge cartridge to be able to fit into two or more different types of centrifuge machines, in order to meet the requirements of this invention. Instead, if a centrifuge cartridges with at least two or more wells that are sized and designed to hold 20 cc syringes is sized and suited to fit in a stable and secure manner into at least one type of conventional desktop centrifuge, then that centrifuge cartridge is regarded as being within the scope of the invention and claims disclosed herein, provided that it meets all other relevant limitations of the claims. Accordingly, one or more manufacturers can sell such cartridges as accessories, which can be purchased by the user of any particular make and model of a desktop centrifuge, who will know exactly which type of desktop centrifuge is present and available for use, in any particular physician's office or clinic. This would be comparable to a typical automobile driver being able to purchase tires for any particular make and model of automobile, merely by telling someone who works at the tire store the make and model of the car that needs new tires.

Alternately, rather than having to address potential issues concerning the various different models of desktop centrifuges that are commonly owned and used by physicians' offices and clinics, a preferred approach to designing and making centrifuge cartridges that will hold at least two and preferably three 20 cc syringes can focus, instead, on the types of specialized centrifuge machines that recently have become available for preparing “platelet rich plasma” (PRP). That class of specialized equipment underwent a major restructuring in 2009, when two specific types of desktop-sized PRP units, designed for physicians' offices and clinics, were commercialized: the SMARTPReP™ system, sold by Harvest Technologies, and the MAGELLAN™ system, sold by the Arteriocyte Company.

Since the preparation of high-quality PRP (with platelet cell concentrations at least 5× greater than baseline, and preferably with reduced granulocyte cell concentrations as well) is essential for the treatments herein, and since both the SMARTPReP and MAGELLAN systems include relatively compact and reasonably-priced desktop centrifuge machines as major elements of those systems, a useful working presumption is that the very large majority of any physicians' offices and clinics which are able to perform the types of treatments described herein will have one of those two types of machines. Therefore, if syringe-holding centrifuge cartridges are manufactured and sold, which are designed and sized to hold at least two 20 cc syringes while fitting into one of those two types of PRP centrifuges, then having those simple options will enable essentially any physician's office or clinic, if equipped to prepare PRP at high quality levels, to centrifuge up to 80 or even 120 cc of liposuction extract at a time, using the methods disclosed herein, in the same desktop centrifuge that is also used to prepare platelet-rich plasma (PRP).

A separate section, below, addresses the dimensions of centrifuge cartridges for PRP machines, and for 20 cc syringes which will fit into those types of cartridges. For now, the sections directly below summarize the major steps of the procedure, with reference to the drawings.

Finally, it should be noted that the ability to centrifuge liposuction extract fluid, while the fluid is still inside in the syringe that was used to extract it rather than transferring it to a different vessel or container for centrifugation, is highly useful and convenient, since it can avoid the requirement for additional steps and safeguards that would need to be developed and utilized, to prevent or absolutely minimize any risk of contamination and infection. Anything which can be done to minimize the number of items or surfaces which will contact any cellular or fluid material that will be injected into a patient, or which can be done to ensure the sterility of any such vessel, tubing, filter, and other device which contacts any cells or fluids that are injected into a patient, can facilitate and improve the types of connective tissue repairs described herein.

### First Centrifugation Step: The Liposuction Fluid

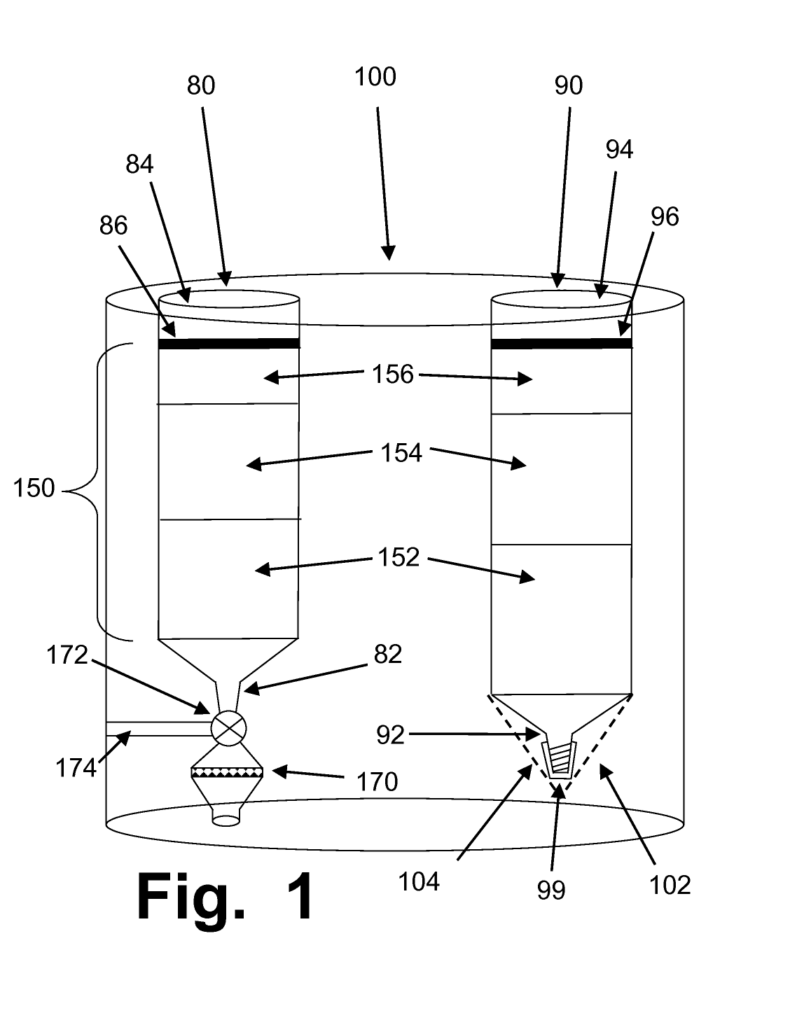

FIG. 1 is a schematic depiction of two syringe barrels 80 and 90, held parallel to each other in a syringe-holding centrifuge cartridge 100. For purposes of description at this stage, both of the two syringes barrels 80 and 90 (which also can be called syringe cylinders, tubes, or similar terms, and which also are referred to herein simply as syringes, for convenience) are standard and conventional disposable plastic “monoject” syringes, as well known in the prior art. However, certain types of modified syringes, having wider diameters and shorter lengths to enable three fully-loaded 20 cc syringes to fit into cartridges that will fit into specialized centrifuges designed for preparing platelet-rich plasma, are also of interest herein, and are described below.

In FIG. 1, each syringe 80 or 90 holds a suitable volume (such as 20 cc) of a liposuction extract 150, taken from a patient in a single liposuction procedure, using methods such as described in Example 1. The liposuction extracts depicted in FIG. 1 have been centrifuged at about 40 G (i.e., at a rotational speed which will establish centrifugal forces that are 40 times greater than the force of gravity) for about 8 to 10 minutes. It has been established and reported, by various researchers, that centrifugal forces of up to about 40 G, imposed for 10 minutes at a time, will not substantially damage or kill the types of stromal precursor cells that are involved.

As depicted in that drawing, the “bottom” (highest density) aqueous layer 152, in each syringe, will contain a watery liquid. This layer will be adjacent to a syringe tip 82 or 92. During high-speed rotation, the syringe tips 82 and 92 will travel around the outer periphery or circumference of the syringe pathway, near the circular interior wall of the centrifuge chamber. The opposed ends 84 or 94 of the syringes are usually referred to as the opening, throat, or similar terms.

The temporary coupling of a syringe tip to a liposuction extraction cannula, during liposuction, can use a conventional “luer” fitting. That name, which came from a 19th century German instrument maker who played a major role in the early development of these devices, refers to standardized fitting sizes that are used for small fluid-handling devices. Luer fittings are divided into two major classes. So-called “luerlock” fittings (also spelled luerlok) use accommodating threads, which are enlarged (compared to typical screw threads), to make them easy to engage, and so that only about 2 rotations are required to fully and securely couple a syringe tip to a cannula or other device.

Other types of “luer” connectors which are not threaded are often called “luerslip” systems. These usually are adequate for small couplings that are used only briefly and that do not need to withstand substantial pressures. However, since any liposuction syringe as discussed herein will need to withstand centrifugation for a prolonged time, luerslip connectors would not be well-suited for such syringes. Accordingly, syringe tip 92 is shown as having luerlock threads, which have been screwed tightly into a small internally-threaded cap 99, for centrifugation. Cap 99 will press securely into a conical or similar “floor” 102, which is provided as part of a well 104 which is designed to hold syringe barrel 90.

Returning to the liquefied material contained in both syringes, the next “higher” layer 154, above aqueous layer 152, contains a thick, viscous, and relatively opaque fluidized or paste-like material, referred to herein as “spun fat” for convenience in this specification, and as “concentrated fatty tissue extract” in the claims. This “spun fat” layer will contain large numbers of cells, along with glycogen, broken collagen fibers, etc.

The uppermost (lowest density) layer 156 will be a generally clear layer of oily liquid, which will contain few if any viable cells. Oily layer 156 will be discarded.

A movable mechanical plunger tip 86 or 96, made of rubber or a flexible polymer, will rest on top of the oily layer 122, near the opening of each syringe. It will form a movable but generally watertight seal between the outer rim of the plunger tip 86 or 96, and the smooth inner surface of the syringe barrel. During the liposuction procedure, a plunger or handle device (not shown), which is small enough to travel within the syringe barrel, will have its lower tip securely attached to a rubber plunger tip 86 or 98, by means such as a threaded fitting; this will allow the plunger handle to be secured to or removed from the plunger tip whenever needed.

During liposuction, the surgeon will exert a gentle but firm pulling force (or a tensile or withdrawing force, or similar terms) on the plunger handle, to create a controlled level of suction inside the syringe, to extract liquefied fatty tissue from the patient's body. When a syringe barrel is sufficiently full of liquefied tissue, the fully-loaded syringe will be unscrewed from the cannula and replaced by an empty syringe, so that more liquefied tissue can be extracted. The surgeon can use as many syringes as desired, for any treatment on any specific patient. If desired, the cannula tip can remain inside the patient, each time a full syringe is replaced by an empty syringe. However, if the surgeon suspects that one or more of the inlet holes of the cannula might be clogged, the surgeon can temporarily withdraw the cannula from the patient (the affected skin area will remain anesthetized and numb), to inspect the cannula and clean it if necessary.

After a syringe loaded with fatty tissue (i.e., fluidized liposuction extract) has been properly centrifuged (such as at about 40 G for 8 to 10 minutes), watery layer 152 will be an aqueous suspension which will contain substantial numbers of stromal precursor cells. The watery liquid itself would interfere with wound healing, and should not be injected into an injury site or other targeted treatment site; therefore, the water will be removed from the stromal cell preparation, and discarded. However, rather than losing and wasting the stromal precursor cells that are contained in aqueous layer 152, the watery layer 152 preferably should be passed through a filter device, which will cause the cells to be effectively held on a filter surface while water passes through the filter and is collected and disposed. Various types of commercially-available cell filters are available, which will allow a watery liquid to pass through the filter while retaining any human or other eukaryotic cells suspended in the liquid. Membrane filters made of polysulfones, with pore diameters that average 13 to 15 microns, have been used by the Applicant herein with good results. Filter segments with suitable sizes (such as discs having 15 mm diameters) can be held in any suitable holding mechanism.

To illustrate the type of cell-filtering devices of interest herein, a filter segment 170 is shown in FIG. 1, embedded within centrifuge cartridge 100, and positioned for use with syringe 80. If filter 170 is embedded within cartridge 100, then at least two additional elements will also be required:

(1) a conduit which will carry the aqueous liquid from the syringe to the filter, after centrifugation;

(2) a valve device 172, which should be positioned between the syringe tip 82 and the filter material 170, to prevent oily material from contacting and coating the filter surface during the centrifugation; and,

(2) a valve access port 174 (or similar means), to allow a physician or assistant to open and close valve 172 at appropriate times.

It should also be noted that if a filter and the necessary conduits and valve are provided within a centrifuge cartrdge, they likely will cramp and reduce the “effective length” of the cartridge, which will need to hold up to three 20 cc syringes.

In addition, the risk that some quantity of oily and/or fatty material, from a liposuction extract, might contact and coat the filter media, before the cell filtering step has been completed, is another potentially important problem. This risk is increased by the fact that if a small “plug” of fatty material is positioned in the narrow tip 82 of syringe 80, it can be difficult to dislodge and displace that “plug” of fatty material, with a slightly denser watery material, no matter how long the liposuction extract is centrifuged, because of the way the fatty “plug” will be effectively forced and “wedged” into a small and narrow channel which will appear to be a “dead end” during centrifugation. Contacting and possibly coating a porous filter with even a small quantity of a thick and sticky oily material, before passing an aqueous solution through the filter, is not desirable, and in some cases it may pose a risk of seriously impairing or disrupting the cell filtering operation.

For those reasons, the filtering arrangement shown on the left side of FIG. 1, while feasible, is not a preferred design, and it is presumed that any cell filtration steps should, instead, be carried out using a separate filtration device which can be optimized for such use rather than being “squeezed” into the tight confines of a cartridge that is designed mainly to hold at least two and preferably three large syringes while spinning in a centrifuge.

Accordingly, syringe 90 (depicted on the right side of FIG. 1) shows a threaded cap 99, which has been screwed onto the threaded tip 92 of syringe 90 during the centrifugation step. During centrifugation, cap 99 will be pressed against a conical sloping (or other suitably shaped) “floor” 102, which will be provided as part of a syringe-holding “well” 104 which is provided in cartridge 100. After centrifugation has been completed, syringe 90 (and cap 99) will be lifted out of the well, a plunger handle will be screwed onto rubber plunger tip 96, and cap 99 will be unscrewed from syringe tip 92. Using the plunger, the aqueous layer 152 will then be pushed out of the syringe barrel 90, and into a filtration device and through a filter material that will allow water to pass through while retaining cells on the surface of the filter.

In one preferred embodiment, the filtration device can be a relatively small and lightweight device which can be turned either “right-side up” or “upside down”, at different stages of the work, to take advantage of gravity-assisted flow during each of two different stages. When a watery liquid containing stromal precursor cells is being passed through the filter, downward flow of the watery liquid, through the filter, is beneficial. However, after that step has been completed, when the time arrives to remove and recover the cells from the surface of the filter so that the cells can be processed for reinjection back into a patient, it becomes beneficial to use a “pulse” of water which travels in a downward direction through the filter. This will allow the washing liquid to dislodge cells from a bottom surface (or underside, or similar terms) of the filter, in a way that will allow the cells to simply fall off the underside of the filter, for collection in a small cup, basin, tray, or similar receptacle that is positioned beneath the filter during the filter-washing and cell-recovery step.

As mentioned below, if a collagenase digestion step will be used, an aqueous saline solution (such as “Hanks balanced salt solution”, abbreviated as HBSS) will be added to the “spun fat” layer, while the spun fat is being incubated with collagenase. Accordingly, that same type of saline solution can be used to wash the cells off a filter surface, when it is time to recover the filtered cells. The saline solution that is used to wash cells off of a filter surface can be used as part of the saline solution that will be added to the spun fat, to help promote a collagenase digestion step.

Alternately, if mechanical processing (rather than enzymatic digestion) will be used to dislodge and detach stromal precursor cells from extra-cellular collagen fibers, then it should be kept in mind that any cells which became suspended in an aqueous layer 152, created by centrifuging a liposuction extract fluid, very likely have already become detached from any extracellular collagen fibers, and therefore will not need to be passed through an additional mechanical processing step, which by its nature will necessarily inflict some level of shearing and other stresses on cells being treated. Accordingly, if a cell-containing filter-rinsing liquid is created, it likely should be held aside and not included, when the mechanical processing step is used to dislodge and detach stromal precursor cells from extra-cellular collagen fibers. The aqueous-suspended filtered cells can be added back to the other cells from the spun fat layer, once the cells from the spun fat layer have passed through the mechanical processing and are ready for final centrifugation.

After the initial centrifugation step involving the “raw” liposuction extract has been completed, to form the layers depicted by FIG. 1, the syringes (with threaded caps still attached to the threaded tips of the syringes) will be lifted out of the centrifugation cartridge, and placed in a stationary holding device, to help separate and process the layers that were formed during centrifugation. Any of several different sequences of steps can be used for the separation steps, such as the following:

(1) the plunger handle is reattached to the rubber plunger tip, which remained at all times inside the syringe barrel;

(2) the threaded cap is unscrewed from the tip of the syringe;

(3) the syringe tip is coupled to a clear conduit or device (presumably a relatively thin flexible plastic tube) that will carry the water layer to a filter device, which will allow water to pass through the filter while retaining the stromal precursor cells on the filter surface. If desired, it may be preferable to divert the first drop of fluid that emerges from each syringe, onto an absorbent material that will be discarded (such as a paper towel), so that if a small quantity of sticky fatty material formed a “plug” in the syringe tip during centrifugation, it will be diverted and disposed of, before it can contact and possibly coat and foul the filter material;

(4) the plunger handle is pushed into the syringe, until the aqueous layer has been expelled from the syringe barrel and passed through a cell filter;

(5) the syringe is then uncoupled from the cell-filtering device which handles the aqueous layer, and is coupled to a different conduit or device;

(6) the plunger handle is pushed deeper into the syringe, to expel the “spun fat” layer from the syringe (it will be a viscous paste-like compound, comparable to toothpaste) and force it into a chamber or vessel that is suited for: (i) a collagenase incubation step; (ii) a mechanical processing step as described below; or, (iii) mixing with a suitable aqueous solution, such as “Hanks balanced salt solution” and/or lecithin or a similar surfactant, before a mechanical processing step as described below;

(7) when the entire “spun fat” layer has been expelled from the syringe, the plunger handle can be pulled out of the syringe (with the rubber tip still attached to it), and the low-density oily layer can be drained and/or rinsed out of the syringe and discarded; alternately, the plunger handle can be unscrewed from the rubber tip, and the syringe barrel, with the oily layer and rubber tip still inside, can be discarded as medical waste.

If desired, the spun fat layer 112 (with a relatively small quantity of filtered cells from the aqueous solution) can be reinjected directly back into the patient, along with platelet-rich plasma. This option is indicated by the lowest box on the left-hand side of FIG. 2, which is a flowchart that summarizes the cell processing steps discussed herein. That treatment method was used by the Applicant herein in a number of early trials, with generally good results when compared to conventional treatments known in the prior art.

However, as indicated by the alternate pathway in lower right quadrant of FIG. 2, a more extensive and thorough processing method involves additional steps which will further enrich and concentrate the stromal precursor cells, before they are injected back into the patient. Those additional steps are described below. Because of both: (i) the nature and the known and predictable results of the additional processing; (ii) the visible results on processed cells, when before-and after results are analyzed under a microscope; and, (ii) results that the Applicant has seen to date in patients who have been tested in small-scale trials of these methods, the Applicant herein regards these additional processing steps as highly beneficial, to a point of clearly justifying the extra time and effort these additional steps require.

### Collagenase Incubation, Followed by a Second Centrifugation

As mentioned above, one approach which can be used to further concentrate stromal precursor cells from a “spun fat” layer involves: (i) incubation with collagenase, followed by (ii) a second centrifugation step.

However, it must also be understood and recognized that if a cell preparation is contacted by a biologically active enzyme, prior to injection of the cells back into a human, major questions of safety and efficacy may arise, and under the laws that apply in the US and essentially all other developed countries, extensive clinical trials are likely to be required to prove, to the satisfaction of the US Food and Drug Administration or comparable agency in any other country, that any cell preparation created by any such biologically-active treatment will not create or suffer from substantial risks of adverse long-term side effects. As an illustration of this principle, the FDA or comparable agencies in other countries would almost certainly raise pointed and persistent questions about the exact extent to which any active collagenase molecules are in fact removed from a final cell preparation, before the cell preparation is injected back into a patient. It can become extremely difficult or even impossible to reliably answer such questions, especially if a “collagenase removal and/or deactivation step” is going to be performed by a nurse or clinical assistant whose training does not match the multiple years of training received by a fully-qualified physician-specialist who graduated from a medical school and then apprenticed for years as an intern and then resident in a teaching hospital. Accordingly, those types of issues, concerns, and problems, which will arise if a cell preparation is treated by an enzyme or any other biologically-active compound, can be avoided entirely, if purely mechanical processing is carried out, using equipment which is either disposable or autoclavable, to guarantee sterility.

In view of those factors, “mechanical treatment” options, as described in more detail below, are generally regarded as preferable to an “enzymatic digestion” option, as described in the remainder of this section.

Collagenase is a mammalian enzyme, which will cleave and break apart collagen fibers. In soft tissues, it is always active, and is part of a constant process of clearing out and removing old collagen fibers (which gradually become degraded, over a span of months, causing their strength and flexibility to become impaired), and replacing them with new collagen fibers, which constantly are being generated and secreted by various types of cells. The constant process of digesting and removing old collagen fibers, and replacing them with new collagen fibers, is directly analogous to the process of apoptosis (programmed cell death) and cell replacement, as described in the Background section.

Accordingly, if collagenase is used to digest the extra-cellular fibrous matrix that will be present but partially broken apart, in fluidized fatty tissues obtained by liposuction, the collagenase treatment can effectively release stromal precursor cells from a relatively sticky and clingy extra-cellular material which can no longer provide any utility or benefits by the time the stromal precursor cell extract has become a centrifuged layer of “spun fat”. Accordingly, a collagenase incubation step, followed by a second round of centrifugation, will effectively convert a mass of sticky “spun fat”, with some cells in it, into a substantially more enriched and concentrated layer or pellet which contains large numbers and high densities of stromal precursor cells.

Accordingly, these additional processing steps point back to the desirability of extracting and processing generous and even large volumes of liposuction fluid, during the liposuction step. As indicated in Table 1, those large volumes of fluid, when properly treated as described herein, will be concentrated into small volumes with exceptionally large numbers of densely concentrated cells that can promote and accelerate connective tissue repair, regeneration, and healing, at the site of an injury or other tissue defect or problem. Accordingly, the disclosure of modified centrifugation cartridges, designed to handle three 20 cc liposuction syringes at a time, which will contain unusually large quantities of fatty tissue extract within limited and constrained barrel lengths, will help surgeons handle and process, quickly and efficiently, the relatively large volumes that are of interest in these particular types of treatments.

If a collagenase incubation step is used, it can use commercially available collagenase, which usually is shipped and stored in dry powdered form. A low-viscosity aqueous liquid should be added to the incubation mixture, at a suitable volume, to function as a solvent and/or suspension agent. The total aqueous liquid in the mixture should be about half of the spun fat volume, and it should be noted that this fractional volume can and preferably should include the quantity of aqueous liquid that was used to wash cells off of the cell filter(s), as described above.

Candidate aqueous liquids that can help promote the collagenase digestion step include a buffered salt solution known as Hank's balanced salt solution (HBSS), which preferably should not contain phenol red or any other indicator-type chemicals. If desired, a small quantity of glucose or fructose diphosphate can be added to the aqueous dilution liquid, to provide the cells with a source of energy, to help them survive the stresses that are imposed on them while they are outside the patient's body. The incubation period should utilize periodic shaking, to promote mixing (such as for 10 to 15 seconds, every 5 to 15 minutes), and the liquid mixture also can be stirred or rocked continuously. Incubation should be carried out at 37° C. for a suitable time, such as 1 hour.

After the collagenase incubation step has been completed, a second centrifugation step should be performed. Since a substantial amount of debris (including glycogen, collagen remnants, and various other types of cellular and extra-cellular debris) will be present, the digestion mixture preferably should be mixed with a substantial or large volume of a cell-free liquid, for the second centrifugation. Bovine fetal serum, and autologous platelet-poor plasma (which can be obtained from the patient's own blood, as a byproduct from the platelet-rich plasma preparation step), have been used by the Applicant herein, with good results, and other candidate liquids (including buffered salt solutions) can also be tested for use in this particular step, if desired. A volume of aqueous diluent (which will effectively perform as a washing liquid, during the second centrifugation step) that can range from about 25% up to about 90% of the total volume of the diluted mixture can be used. This volume of liquid is not crucial, since the added aqueous liquid will simply be removed again by the centrifugation step. However, the low-viscosity aqueous liquid can facilitate and enhance the second centrifugation step, by reducing the tendency of collagenase and/or debris to interfere with the cells, as the cells (which have the highest density) are driven toward the outer tip of the tube, cone, or other container that holds the mixture during centrifugation.

Because of the volumes that will be involved, any cartridges that will be used for this centrifugation step preferably should not be subdivided into chambers, syringe wells, etc.; instead, the entire interior volume of any such cartridge should be “usable”. It generally is preferable to provide a cone-shaped “bottom” for these types of centrifuge cartridges, to cause the pellets to be compacted into a “pellet” with a reduced size, rather than being distributed as a layer across the entire bottom of a flat-floored cartridge.

Accordingly, after centrifugation is carried out at a speed which will not damage the cells (such as at 40 G), for a sufficient time to achieve separation (which presumably will be about 8 to 10 minutes, in most cases), the supernatant can be discarded, and a compacted cell pellet or layer will be ready to be mixed with platelet-rich plasma (PRP), for injection into the patient.

Alternately, if desired, the cell pellet can be stored in a freezer for some span of time. The maximal limits for storage, without damaging the cells, have not been tested; however, cell storage for up to six months, at −80° C., has been tested, and no significant damage to or deterioration of the cells was detectable, when analyzed by methods that are designed to detect nonviable cells.

It should be noted that certain claims contain limitations which refer to, “removing at least a substantial portion of the supernatant, from the layer or pellet of cells.” That phrase is intended to reflect the simple fact that it normally does not require additional steps, or a high degree of care or precision, to effectively isolate a layer or pellet of cells from a supernatant, or to treat any centrifuged fluid in a manner that exploits precise boundaries between the layers. Exact and precise boundaries, between different layers of a biological fluid, usually are not created when a centrifugation process must be kept to relatively low speeds and limited times in order to protect the viability of living cells. Therefore, when a layered fluid generated by a centrifugation process is being decanted, siphoned, or otherwise processed to separate the layers, the typical practice in most clinical settings is to isolate the layer of interest, plus a relatively small zone of “transitional layers” both above and below the layer of interest. Suince this practice is not exact and precise, the best way to describe it, in language suited for a patent claim, is to simply assert that “at least a substantial portion” of the unwanted layer(s) are removed, from the layer or pellet of cells which are being isolated.

### Mechanical Processing, to Generate Shearing Forces that will Detach Viable Cells from Extra-Cellular Collagen and Fat

As mentioned above, if a biologically-active compound (such as collagenase, an enzyme) is used to treat cells which will later be injected back into a human, questions concerning safety and efficacy will arise. Such questions are likely to require extensive clinical trials, to generate statistical data that will be sufficient to prove, to the satisfaction of regulatory experts and agencies, that any such treated cell preparations remain safe and effective, after the biochemical treatment.

By contrast, if mechanical processing is used, in which the cells are contacted during such processing only by reliably sterile surfaces (which can be ensured by means of proper clinical procedures, using devices that are either disposable or autoclavable), potentially problematic questions (such as whether collagenase molecules have been fully and adequately removed from a cell preparation) will not arise, and extensive clinical trials will not be required. Accordingly, a preferred method of quality control, when this type of mechanical-only cell processing is done, is to simply check a small sample of the cells that are present in the final pellet or layer of cells, using a light microscope, to ensure that the large majority of the cells remain intact and viable, and have not been ruptured, lysed (i.e., broken) or otherwise killed or damaged.

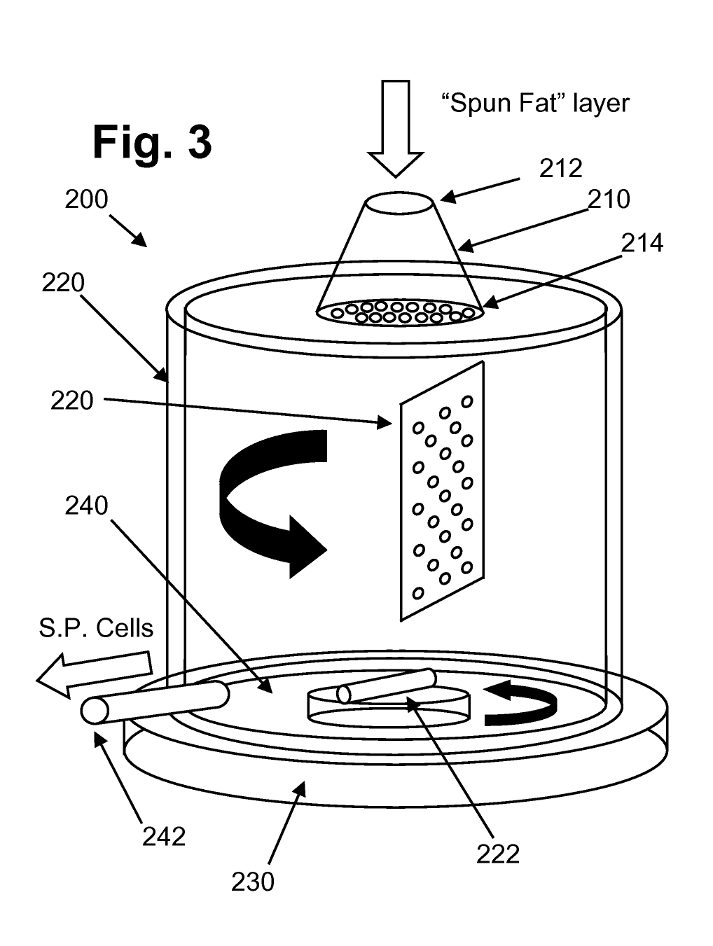

Accordingly, FIG. 3 illustrates an extruder device 200 that can be used to separate viable stromal precursor cells from the extracellular “matrix” (composed mainly of fat, collagen fibers, an oily liquid, and a watery liquid) that will be present in a “spun fat” material created by centrifuging a liposuction extract.

Prior to inserting the spun fat material into the extruder 200, it should be mixed and diluted with a suitable buffered watery liquid (such as HBSS, mentioned above), to render it less viscous, and to help make it easier for the cells to separate and disengage from the relatively sticky materials in a spun fat layer. If desired, a compound such as lecithin (a relatively gentle surfactant mixture, which is already approved for contacting and treating cells that will be returned to a patient's body) can also be added to that mixture.

The cell and fat mixture is loaded into extruder 200, which generally has a cylindrical shape and which can be made of a transparent material that can be sterilized in an autoclave after each use (such as clear polycarbonate), via an inlet device 210, which is positioned on top of extruder 210 to enable gravity to assist in the flow and separation process. The inlet device should be provided with a pressure-tight inlet connector 212, such as a threaded “luerlock” fitting that will accommodate a syringe having a luerlock outlet.

The cell-plus-fat mixture is forced, by fluid pressure applied by the operator (such as by pushing a syringe plunger into a syringe barrel) to pass through an extruder plate 212, which will have multiple small holes passing through it. Early tests have indicated that extruder holes having a conical shape, with an inlet diameter of 3 mm and an outlet diameter of 2 mm, passing through a plate having a suitable thickness (such as 3 to 10 mm), provide very good results.

After the diluted cell-and-fat mixture enters the main cylindrical chamber 220, it will begin to rotate, as indicated by the arrows, driven by a powered stirring mechanism 222. A conventional magnetic stirring rod 222 is illustrated, for simplicity; if desired, a more elaborate and powerful device can be used, such as a device having two or more vertical stirring paddles attached to a magnetically-drivable base.

As the watery-fatty cell suspension circles through the chamber 220, it will pass through a set of perforated “catch plates” 224. Because of surface tension factors, globules and droplets of fat and oil that are suspended in the moving watery solution will stick and cling to the catch plates, whenever they contact a surface of one of those plates. This will help “clear out” the mixture, and make it easier for the cells to be separated cleanly.

For simplicity of illustration, only one catch plate 224 is shown, in FIG. 3. In actual practice, a set of 3 catch plates, mounted radially at 120 degree intervals around the outer wall of chamber 220, has provided very good results.

The base unit 230 preferably should be provided with a heating element, to help ensure that the suspension remains at a preferred temperature during the entire cell separating process. Since a temperature range of about 105° F., up to about 110° F., will emulate a fever which would be very severe for a living human (especially for brain tissue), but which will not rapidly kill the types of stromal precursor cells of interest herein, suitable temperature ranges for optimization testing as disclosed herein begin at about 103° F., and likely will range up to about 110° or possibly even higher.

Over a span of time, which in early tests has ranged from about 5 to about 20 minutes, the freed and released cells will descend to the bottom floor 240 of chamber 220. Bottom floor 240 preferably should have a sloping surface, to help convey the cells to a valved outlet port 242. If the walls of the chamber 220 are made of transparent material, the person running the separation process can monitor it visually, both to ensure that everything is moving and proceeding properly inside the chamber, and to determine when the liquid reaches a state of sufficient clarity and transparency to indicate that the very large majority of the cells, initially suspended in the solution, have dropped out of solution and are ready to be removed from the chamber.

If desired, other types of treatments (which most commonly include increased increased salt content, increased acidity, etc.) that are used to release affinity-bound molecules from sorbent materials, during affinity purification procedures, can also be tested, to determine their ability to help detach and separate the desirable stromal precursor cells, from any the unwanted extra-cellular debris (including collagen fibers, glycogen, etc.) that is present in a “spun fat” mixture.

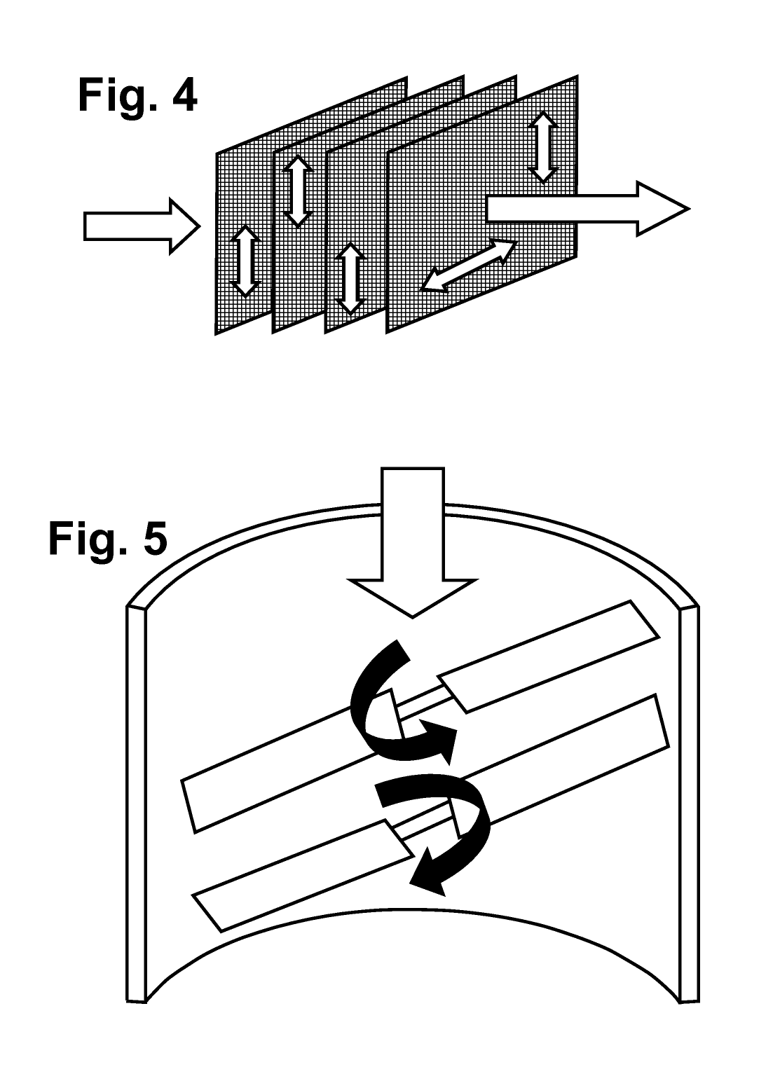

FIGS. 4 and 5 provide simplified schematic depictions of two alternate mechanical processing devices, which show other types of mechanisms that can be used, if desired, to detach viable stromal precursor cells from extra-cellular collagen and fat in a “spun fat” layer, in ways that will not damage or kill the cells.

### Passage of Spun Fat Materials through Screen Devices

For example, as depicted schematically in FIG. 4, passage of spun fat through one or more vibrating or hammered screens can accomplish the same result as an extruder device. The distinction between “screens” and “extruders” is not precise, and devices can be created which are at a halfway point between those two classes of devices. If that situation arises, either term can be applied, depending on which term makes more sense when applied to a particular device. In most situations, a device would be referred to as a screen, rather than an extrusion device, if either of the following conditions apply:

(1) if it is made from fibers, strands, or wires (usually made of metal wires, or strands of nylon, polyethylene, or a similar polymer) that have been woven together or otherwise interlaced, rather than (i) using pre-molded pieces which have holes or orifices already in them, or (ii) using punching, drilling, or other types of machining to create holes through a pre-existing layer or sheet of material; or,

(2) if it is made from a relatively thin layer or sheet of material which is designed to be flexible after the holes have been created in it, and if the area occupied by the holes comprises at least half of the “working area” of a sheet of such material.

Either set of conditions mentioned above will normally lead to an operating requirement that a screen must be kept under tension, when being used as a “cell passaging” component of a fluid-handling system, to prevent it from being severely distorted when the fluidized meterial passes through it. To meet that requirement, replaceable cartridge-type devices can be created, with each “cartridge” containing a segment of screen with its outer edges embedded in a relatively thick frame or border made of rubber or a flexible polymer. A series of such cartridges can be inserted into corresponding slots which are positioned, with appropriate spacing between them, inside a flow conduit.

Alternately, rather than using replaceable screen cartridges, a screen-separation conduit can be manufactured as a disposable single-use unit, made of relatively inexpensive plastic with one or more screen segments embedded within the device.

Regardless of whether a screen-passaging unit uses replaceable screen segments or is manufactured as an integrated unit, one of the benefits of using two or more segments of screen (rather than an extruder device) to isolate stromal cells from a “spun fat” material is that the different screens, in a series of two or more screens, can have different design features, such as different hole sizes. It is presumed and believed by the Applicant herein that passage of a spun fat layer through a first screen having moderately large holes (such as between 0.8 and 1.5 mm in width, when square holes in a woven screen are being used), and then through a second screen having smaller holes (such as 0.3 to 0.8 mm widths), is likely to enable higher levels of cell separation and isolation, with minimal or at least acceptable levels of cell damage, compared to extrusion, or to screen passaging using only a single screen having fixed dimensions.

Passage of a spun fat material through three or more screens, with each screen containing slightly smaller holes than any prior screen, may be able to promote even higher levels of cell separation and isolation. However, each and every passage of the cells, through a flow obstacle such as a screen, will inevitably impose substantial levels of stress on the cells that are being treated. Therefore, keeping the number of such passages to a minimum will help reduce cell mortality.

Accordingly, unless and until data from optimization testing indicates otherwise, it is presumed that passaging of spun fat through two (and only two) screens is likely to achieve the best balance between maximal cell separation, and minimal cell damage, provided that both screens have dimensions which have been determined by a systematic testing program that generates and then evaluates cell separation and cell mortality curves, as a function of a range of screen dimensions.

As an initial teaching and disclosure which is believed to enable the effective use of double-screen cell passaging, and which is also believed to offer a set of useful and practical dimensions for commencing a testing program that will determine optimal screen dimensions for double-screen passaging systems, it is asserted by the Applicant here that initial screen sizes of 1 mm hole widths for the first screen, and 0.6 mm hole widths for the second screen, can provide useful and effective cell-separating results for spun fat material from liposuction extracts, when both screens are woven from round strands of a polymer such as nylon or polyethylene, with the strands having diameters of at least about 0.2 mm.

If a testing program is commenced to determine truly optimal design and operating parameters for the use of screen-passaging to separate stromal cells from extra-cellular debris in “spun fat” from a liposuction extract, then each of the following parameters will merit serious consideration, for treatment as a controllable variable which can be evaluated to determine its ability to help maximize cell separation while minimizing cell mortality.

1. optimal hole sizes, for both (i) a single screen, and (ii) each screen, if two or more screens will be used;

2. optimal hole shapes. If an “orthogonal” weaving pattern is used, it will create square or rectangular holes, with corners that nominally have consistently 90 degree angles. More complex weaving patterns can be used, if desired, which can create, for example, a honeycomb pattern with hexagonal holes which do not have the relatively “sharp” corners of square or rectangular holes.

It should also be noted that the “nominal” 90 degree angles of the corners of square or rectangular holes, and the “nominal” 120 degree angles of hexagonal hole junctures, do not accurately indicate what will be actually encountered by cells that are passing through a screen. Even though the “nominal” angles of the square holes in a conventional screen are 90 degrees, the actual angles which will be created, by the interweaving of cylindrical strands which typically will have diameters at least 10 to 20 times greater than a cell diameter, will be much more complex, with a number of small acute wedge-shaped angles at each and every intersection of two strands in the screening material. Those miniature acute angles are likely to help promote better cell separation, even at slow cell-travel speeds, when a screen is used as described herein, and their effects on cell damage and mortality are likely to increase at increasing rates (possibly even approaching exponential increases), as cell-travel speeds are increased. Therefore, a range of cell-travel speeds (which can be controlled and modulated, by controlling pressure gradients and pumping rates) will need to be evaluated, when any particular screen dimensions and weaving pattern are being evaluated.

Alternately, those types of factors and issues can be avoided (or at least shifted to greatly reduced levels of significance) by: (i) creating a screen from a sheet of foil or similar material, and then (ii) punching, drilling, laser-cutting, or otherwise treating that sheet of material, to create an array of holes through the sheet. The holes can have any desired size, and any desired pattern (the most common patterns are square grids, and “honeycomb” patterns). However, it must be noted that whenever a machining process is used on a pre-existing sheet of material, it may create a set of “directional” traits in the treated sheet. For example, if a punching or drilling process is used, the “top” side tends to have slightly rounded depressions surrounding each hole, while the “bottom” side of each hole is likely to be surrounded by small irregular spurs or fragments of material, protruding outwardly from the main sheet. Accordingly, if a screen is created by machining an already-formed sheet of material, the “direction of machining” should be recorded, and tested, as a potentially significant factor when the screen is used for separating cells from extra-cellular debris in a spun fat suspension.

3. If a cell-separation screen is made from woven strands, the strand thickness(es) become a design and operating parameter which can be controlled, and which should be tested and optimized. If the strands are too thin, they can cut open and kill cells, in a manner comparable to a cheese slicer which uses a thin wire to cut through a block of cheese. This concern is aggravated by the fact that the stromal precursor cells, in a spun fat suspension, will not be surrounded by a watery liquid which will allow the cells to simply move aside and flow around an obstacle they encounter; instead, the cells will be surrounded by, and effectively embedded within, a thick, sticky, highly viscous mass of fatty semi-solid material from which most of the water and oil has already been removed, rendering the “spun fat” even thicker and more viscous and sticky than normal fat.

Strand diameters, in a material used to make a cell-separating screen as described herein, can be expressed as a multiple of the cell diameters. Because of the factors discussed above, a presumption arises that: (1) the strands should be made from monofilaments (either polymers, or metal wires) with smooth cylindrical surfaces, rather than from woven or braided strands of even smaller fibers; and, a good starting thickness, for optimization testing, can be provided by strands which have thicknesses (diameters) that are about 20 times greater than the relevant cell diameters. Since the diameter of a typical non-fibrous eukaryotic cell is roughly 10 microns (i.e., 1/100 of a millimeter), this implies that a good starting thickness, for optimization testing of strands that will be used to make cell-separation screens, will be in a range of about 200 microns, or 0.2 mm.

4. The number of screens, the linear distance which separates the screens, the “linear flow” rates and speed of cell travel through the screening conduit, and the pressure gradient between the inlet and the outlet of a screen-passaging chamber, should all be treated as a controllable design parameters, in any optimization testing.

5. The ability to impart vibrational, reciprocating, or other motion, to any or all of the screens, must also be considered as a design parameter that can be controlled and then evaluated. As described above in relation to extruder devices, such motion can be a sinusoidal-type vibration, or a tapping, hammering, or other jarring motion. In either case, frequency and amplitude can each be adjusted, and then evaluated.

6. In addition, as described above for extruder devices, various known methods can be used to introduce any one or more of several different types of energy inputs (such as sonic or ultrasonic waves, physical vibrations, or electrical fields) into a cell suspension that is being passed through one or more screen-separators. Similarly, a stirring paddle or similar device can be used to stir or agitate the cell suspension, or to otherwise subject a fat-and-cell mixture to shearing forces just before the cells pass through a screen.

### Shearing-Force Agitation of a Spun Fat Material

FIG. 5 is a simplified depiction of a fluid-handling system which will force a spun fat preparation through a conduit in which rotating, reciprocating, or otherwise moving paddles (which can also be called agitators, blades, or similar terms) are used to generate shearing forces that will help detach cells from extra-cellular collagen fibers and debris.

FIG. depicts two sets of paddles, mounted on a central vertical axle (not shown), which are angled, and which are rotating in different directions, to generate shearing forces in the turbulence they will create. Any number of such rotating paddles can be used, and they do not all need to be mounted on a single centered axle; for example, a paddle system can be designed with effectively interlocking “fingers” which will “sweep” across an enclosed area, in which the “even-numbered” and “odd-numbered” paddles will be moving in opposite directions at nearly all times, except when they pause to change directions. These are matters of relatively straightforward design, and they can use sophisticated stirring systems which already have developed for either or both of two fairly common uses: (i) manufacturing of liquid mixtures which contain ingredients that do not mix well together naturally, but which must be mixed together very thoroughly, to ensure consistent quality of the complete mixture; and, (ii) storage of liquids that need to be kept mixed together thoroughly.

If those types of devices are used to generate shearing forces that will help detach stromal cells from extra-cellular debris in a spun fat material, the design and operating parameters which should be evaluated, during any optimization testing, including the following:

1. The dimensions and other traits of the paddles which will be used, including their overall shape, their thickness, the shapes and countour sof their edges, and the presence, size, spacing, and countours of any holes that are present in the paddle surfaces;

2. The slanted angles, rotational speeds, spacing, and placement of the paddles, within a stirring chamber;

3. The average linear speed of the cell suspension which is traveling through the stirring chamber; and,

4. whether (and to what extent) any additional energy or other inputs (such as sonic or ultrasonic waves, vibrational motion, electric fields, etc.) will also be introduced into the stirring chamber while the cells pass through it.

As a final comment regarding any such optimization testing, it can be performed, using actual cells and entirely realistic conditions, by using either or both of:

(1) centrifuged “spun fat” cell suspensions obtained from humans, via liposuction that was carried out for weight loss purposes, when the quantity of fat that is removed in a single operation is typically measured in liters, rather than milliliters; and/or,

(2) processed and centrifuged cell-containing fatty tissues obtained from cows, pigs, or other livestock that are processed at a slaughterhouse or rendering plant.

### Dimensions for 20-CC Syringes that Will Fit into Standard Centrifuge Cartridges for PRP Machines

The Applicant herein uses a SMARTPReP™ system, sold by Harvest Technologies, so the dimensions discussed herein are based on measurements of that system. It is believed that the relevant components of the MAGELLAN system have comparable dimensions, and can be adapted accordingly for use as disclosed herein.

The SMARTPREP centrifuge has a single rotor, with two opposed and balanced “arms” mounted on opposite sides of a vertical axis which rotates at high speed. Each arm of the rotor holds a “cup” (which can also be called a basket, holder, cartridge holder, or similar terms) at the outer end of the rotor. The two cups are diametrically opposed to each other, for proper balance and minimal vibration during high-speed rotation.

Each cup is mounted at one end of the rotor arm, by means of two pins on opposite sides of the cup. Those two pins, and accommodating support mechanisms in the rotor arms, interact to form a rotatable support for each cup. This allows the “bottom” of each cup (and the bottom of a cartridge held by a cup, and the bottom of a syringe held by a cartridge) to rotate outwardly, into an essentially horizontal position during high-speed rotation, due to the centrifugal forces which will be imposed on the cups, cartridges, and syringes. As mentioned above, centrifugal forces should be limited to about 40 G, to avoid damage to the cells.

The following discussion, concerning the dimensions of the cartridges and cups in a PRP centrifuge, arises from the fact that for convenience and speed, it generally is preferable to be able to use a single desktop centrifuge for both of two different types of centrifugation steps (i.e., (i) for centrifuging syringes that contain liposuction fluid; and, (ii) for centrifuging blood, in order to isolate PRP), without requiring any delays or alterations in the machine, between those two steps.

However, it should be recognized that the standard rotor arm, in a centrifuge designed for PRP isolation, can be removed and replaced by a different rotor arm, relatively quickly and without requiring any specialized tools, merely by: (i) unscrewing and removing a specialized retainer cap, from the top of the vertical axle which supports the rotor arm; (ii) lifting off and removing the standard rotor arm from the vertical axle; (iii) replacing the standard rotor arm with a different rotor arm which can have a shorter length if desired; and, (iv) replacing the retainer cap on the axle, in a manner which secures the new rotor arm to the axle.

Therefore, longer and deeper rotor cups can indeed be used, to hold and support longer and deeper cartridges that are designed to hold standard-sized 20 cc syringes. This can be accomplished, fairly easily, merely by removing a standard rotor arm, and temporarily replacing it with a shorter rotor arm which will allow a deeper cup to be used without the bottom of the cup approaching too closely to the inner wall of the centrifuge chamber.

If it is decided to not use a shorter rotor arm, to enable the use of deeper cups and longer cartridges, then careful attention will need to be paid to the dimensions of the cups, cartridges, and syringes that will be involved during centrifugation of a fluidized liposuction extract.

The standard cups that normally are contained in a SMARTPReP centrifuge have internal diameters of about 3 inches (about 7.6 cm), and depths of about 3.4 inches (about 8.6 cm).

A widely used and standardized type of 20 cc syringe (made of inexpensive plastic, and designed to be discarded after a single use, to avoid risks of contamination) has an internal diameter of 1.9 cm, and a total length of 10 cm when the plunger handle has been removed. Because of the “headroom” that is available, which normally allows the cups and cartridges to swing into an outwardly horizontal position when the rotor begins to spin, it is believed that those types of standardized 20 cc syringes will be able to fit into specially-adapted centrifuge cartridges that will fit into a SMARTPReP centrifuge. However, that will create a “tight fit”, which will constrain and limit the thicknesses (and therefore the strength and durability) of the “walls” and “floors” that are used to make such a centrifuge cartridge.

To provide a more “comfortable and convenient” system (rather than a crowded and compacted system that can barely fit into the space that is available), slightly deeper centrifuge cups can be provided, which can utilize some portion of: (i) about ¼ inch of “gap” space that exists, between the bottoms of the standard cups, and the inner wall of a centrifuge chamber, and/or (ii) about ½ inch of “headroom” space, in the area where the centrifuge cups are mounted to the ends of the rotor arms.

Alternately or additionally, somewhat shorter syringes, with syringe barrels having internal diameters wider than the 1.9 cm which is used in the standardized syringes described above, can be provided.

When accommodations are made for wall thicknesses (assuming at least 2 mm wall thicknesses for centrifuge cartridges that will be reused, and 1 mm wall thicknesses for syringes), there is sufficient room within a standard centrifuge cartridge (having an internal diameter of 3 inches, or about 7.6 cm) for three syringes, with each syringe having an internal diameter of up to about 2.6 cm.

A standard 20 cc syringe with an internal diameter of 19 mm (radius=9.5 mm) requires a calculated length of 7.035 cm to hold 20 cc of liquid; however, when the additional volume of liquid that will be contained in the syringe tip is also included, the measured length, from the inside “shoulder” surface of the syringe to the 20 cc marking line on the barrel, is only about 6.4 cm. The remaining 3.6 cm of syringe length is occupied by the tapered tip (about 1.2 cm), and the opening or “throat” portion of the syringe (about 2.4 cm).

By contrast, if a 20 cc syringe were to be manufactured with an internal diameter of 2.6 cm (radius=13 mm), it would require a calculated length of only 3.77 cm, rather than 7.035 cm as in a standard syringe, to hold 20 cc of liquid. Accordingly, if the tip and throat lengths were unchanged, a syringe with 2.6 cm internal diameter could be reduced, in total length, from 10 cm for a standard syringe, to about 6.7 cm for the modified syringe.

However, a syringe that short, wide, and “stubby” likely would not feel normal or “comfortable” in the hands of most physicians who perform liposuction. Since liposuction is an invasive procedure, in which “feel” and tactile sensations play important roles in helping a surgeon or physician remove fatty tissue without damaging surrounding tissue, a jump from 19 mm to 26 mm, in syringe diameter, would not be optimal. Instead, smaller increases in diameter, such as to about 20 to 22 mm in internal diameter, are regarded as preferable. If a syringe has an internal diameter of 21 mm, which is only 2 mm wider than a standard 20 cc syringe, it will require only 5.26 cm of length to hold 20 cc, compared to 6.4 cm for a standard syringe. That reduction in length, of nearly 1.4 centimeter compared to a standard 20 cc syringe, can provide ample clearances in all dimensions and directions.

Similarly, an increase in internal diameter of a single millimeter, from 19 mm (standard) to 20 mm (modified), could provide a reduction in overall length of about 6.7 mm, compared to a standard 20 cc syringe. That relatively modest reduction in length likely would be sufficient to provide a reasonable balance between “clearance” and “comfort”, for syringes that will be short enough to fit into cartridges that will fit into PRP centrifuges such as the SMARTPReP system, in a manner that will enable two centrifuge cartridges to hold a total of six 20 cc syringes (three syringes in each cartridge) in each “run”.

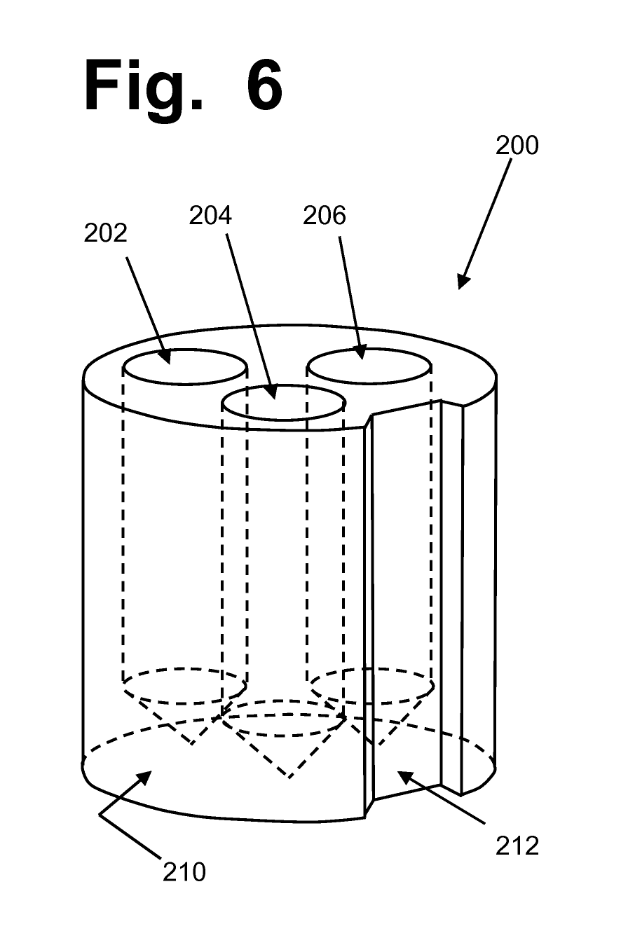

Accordingly, FIG. 6 is a perspective view of a centrifuge cartridge 200, which is provided with three wells 202, 204, and 206, which are sized to hold a 20 cc syringe in each well. The outer cylindrical wall 210 of cartridge 200 is also provided with a “notch” 212. This notch is sized and designed to interact with an accommodating protrusion (often called a “key” or similar terms) in the inner wall of a centrifuge cup. The notch must be properly aligned with the key, in order to insert the cartridge into the cup. This system, which is standard in PRP centrifuges, helps prevent and minimize any vibration, rotation, rattling, or other unwanted motion of the cartridge, within the cup, during high-speed centrifugation.

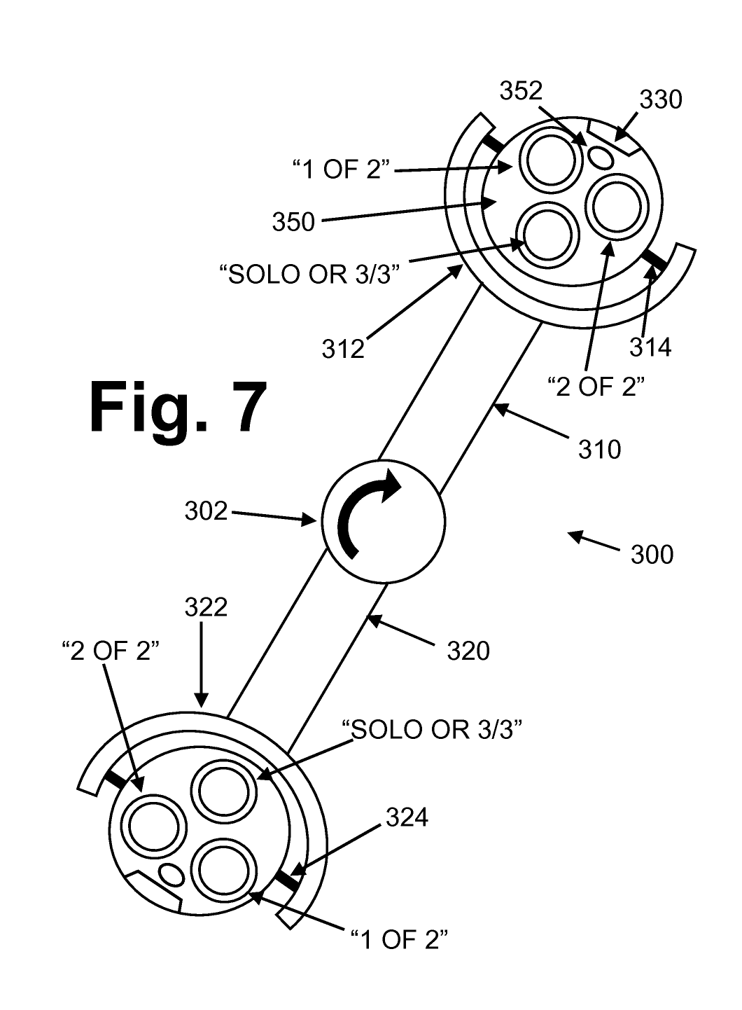

FIG. 7 is an overhead (plan) view of a centrifuge rotor 300, showing a centrifuge cup 330 and a syringe cartridge 350 resting in each of two support mechanisms 312 and 322, positioned at the ends of rotor arms 310 and 320. The rotor arms 310 and 320 rotate in a horizontal plane, around a vertical axle 302 which is coupled to a motorized drive unit (not shown). To simplify the drawing, it is assumed herein that each cartridge has a “lip” around its circumference, which rests upon and is supported by the rim of the cup; therefore, the only portion of each cup 330 which is visible from above is the “key” portion of the cup, which fits into the notch (shown by callout number 212, in FIG. 6) of a syringe cartridge 350.

Each support mechanism 312 or 322 has a pair of support pins 314 or 324, positioned on opposite sides of the centrifuge cup 330 held by the support mechanism 312 or 322. Each pair of pins 314 or 324 enable the cups and cartridges to rotate around a horizontal axis created by the paired pins; this allows each cup-and-cartridge unit to swing into a “horizontally outward” orientation, where the “bottoms” of the cups and cartridges closely approach the inner wall of the centrifuge chamber, as the rotational speed of the centrifuge rotor increases until the centrifugal force exerted on the cups and cartridges exceeds the force of gravity.

Any of several approaches can be used to ensure that syringes loaded with liposuction fluid are placed in the two offsetting centrifuge cartridges in a balanced and symmetric manner, regardless of the number of loaded syringes that are involved. These options include labeling the tops of the cartridges with information to establish the preferred loading sequence, as indicated by the remarks in quotes shown in FIG. 7. If only two syringes will be centrifuged (i.e., with one syringe at each end of the rotor), they should each be loaded into a well that is marked with a phrase such as “Solo or 3/3”. That well should be positioned along the “centerline” of the rotor arm. If four syringes are being centrifuged, they should be loaded into the four wells that are labeled as “1 of 2” and “2 of 2”, to maintain balance and symmetry. If six syringes are being centrifuged, then they must and will occupy all six wells. Accordingly, if that loading sequence is used, balanced and symmetric weighting will be sustained, regardless of whether two, four, or six syringes are being centrifuged. This will avoid placing any non-symmetric stresses on the rotor, or on the supporting pins or other components. While the amount of unbalanced stresses that would be imposed on the support pins and cups (and on the machine), if a different loading sequence is used, would not be great, and almost certainly would not damage the machine in any particular usage session, those types of stresses arising from off-balanced loading should not be imposed repeatedly on a machine, because they would create unwanted and unnecessary stresses that could well lead to unwanted wear and degradation of various bearings, couplings, and other components, over a span of months or years.

It should also be noted that balancing weights, such as syringe-type barrels filled with water or any other fluid or other weights, can also be used to offset and balance out any syringes that have been loaded into a centrifuge. These types of “counterweights” are conventional, and are routinely used whenever a single loaded tube, vessel, or other container (or an odd number of loaded tubes or containers) would otherwise create unbalanced loading, during centrifugation.

Indeed, in order to ensure that closely-balanced weightings will used for each and every “run” in a centrifuge machine, it is deemed to be advisable and preferable to provide physicians' offices that will be using this system with a small and convenient mechanical balance (which can also be called a “balance scale” or similar terms). This type of scale can have a balance arm mounted on a centered pivoting device, having cups at each end that are shaped identically to the cups in a centrifuge, and with a needle or pointer in the middle of the balance arm, which will point vertically toward a “Proper balance” indicator when the balance arm is exactly horizontal. A small additional well 352 is shown, positioned on the “midline” of each cartridge 350, for balancing purposes. If a balancing operation indicates that two fully-loaded cartridges have unbalanced weights, a small quantity of water can be added to well 352 of the low-weight cartridge, until the weights of the two loaded cartridges are exactly balanced.

### Aspects of the Invention

As set forth above, this provisional application describes an interconnected set of devices, machines, and methods for: (1) using minimally-invasive liposuction methods to extract stromal precursor cells from a patient who needs connective tissue repair; (2) processing the liposuction extract to create a concentrated preparation of stromal precursor cells; and, (3) reinjecting the stromal precursor cells back into the patient, along with platelet-rich plasma, at a site where connective tissue is damaged or defective.

Accordingly, the Applicants have identified each of the following aspects of this invention as comprising apparently new and patentable inventions, and it reserves the right to use this provisional filing to establish a priority date for utility patent applications that may cover any or all of the following patentable components or aspects of this set of devices, equipment, and method.

One class of devices that are disclosed herein, and which are believed to be new and patentable, are certain new types of centrifugation cartridges. The Applicants herein have disclosed a new type of cartridge, properly sized for holding syringes in a centrifuge cup during centrifugation, wherein the cartridge:

a. has external dimensions which allow it to be placed within and held securely by a centrifuge cup in a conventional centrifuge machine of a type found in physicians' offices;

b. has at least two (and preferably three) wells, each of which is capable of holding and supporting a disposable syringe that contains 20 cubic centimeters of fluid obtained from a patient via liposuction;

c. has sufficient strength and reinforcement to enable repeated use of said cartridge for centrifugal processing of syringes containing fluid obtained from patients via liposuction, at rotational speeds which generate centrifugal forces that are at least 40 times the force of gravity.

In one preferred embodiment, since any physicians' office that will be performing the types of procedures described herein will need to have a centrifuge unit which is suited for preparing platelet-rich plasma from the blood of the same patient who is being treated, the cartridge can have external dimensions which allow it to be placed within a centrifuge cup in a centrifuge machine that is designed and suited for preparing platelet-rich plasma.

Another class of devices which are disclosed herein focus on more complete centrifugation systems, which include both a centrifuge machine, and a plurality of cartridges as disclosed herein. These claims make no effort to claim the machines themselves, when sold and used without these cartridges, since those machines clearly and already commercially available, and are prior art. Nevertheless, when those machines are combined with the new types of cartridges and cell-filtering devices disclosed herein, the combination of such machines with the new and additional devices creates a new type of system which can be used to perform exceptionally useful and effective medical treatments that were not previously possible on a practical and efficient level that can be carried out in settings such as out-patient clinics.

Accordingly, those types of expanded and enhanced centrifugation systems include:

a. a centrifuge machine of a type found in physicians' offices, which is designed and suited for preparing platelet-rich plasma from blood, and which has two centrifuge cups at opposed ends of a rotor, wherein each centrifuge cup is designed and suited for holding and accommodating a removable cartridge during centrifugation of a body fluid;

b. a plurality of centrifugation cartridges, each of which is sized and designed to fit within a centrifuge cup in said centrifuge machine, wherein each centrifugation cartridge contains at least two wells, with each well being sized and designed to hold and support a disposable syringe that can hold 20 cubic centimeters of fluid; and,

c. at least one filtration device containing fluid handling components and a filter that allows aqueous liquids to pass through said filter while retaining eukaryotic cells on at least one surface of the filter.

In addition to those devices and systems, this provisional application discloses a new method for treating connective tissue defects. This method comprises the following steps:

a. removing (preferably by minimally-invasive liposuction methods) a liquefied fatty tissue extract containing stromal precursor cells, from the patient who will be treated;

b. centrifuging the fatty tissue extract, to separate it into an aqueous layer, a layer of concentrated fatty tissue extract containing viable cells, and an oily layer;

c. treating the concentrated fatty tissue extract, by means of either mechanical processing or collagenase digestion, in a manner which to causes viable cells to be released from extracellular collagen fibers in the concentrated fatty tissue extract;

d. centrifuging the treated fatty tissue extract, in a manner which creates a concentrated preparation (such as a layer or pellet) of stromal precursor cells; and,

e. injecting the concentrated preparation of stromal precursor cells, combined with platelet-rich plasma from the same patient, into a site in the patient's body that is in need of connective tissue repair.

Preferably, the aqueous layer from the liquefied fatty tissue extract should also be passed through a cell filter, to retain cells suspended in the aqueous layer while allowing the watery liquid to be removed and disposed of Those cells can be washed off of the filter, and added to the digestion mixture containing collagenase and concentrated fatty tissue extract.

In addition to claiming that method of treatment, a product-by-process composition of matter is also one aspect of this invention. This product-by-process composition comprises a cell preparation for treating connective tissue defects, created by the following steps:

a. removing a liquefied fatty tissue extract containing stromal precursor cells, from a patient with a connective tissue defect;

b. centrifuging the liquefied fatty tissue extract at a speed and for a duration which separates the liquefied fatty tissue extract into an aqueous layer, a layer of concentrated fatty tissue extract containing viable cells, and an oily layer;

c. treating the concentrated fatty tissue extract, using either mechanical processing or collagenase digestion, in a manner which releases viable cells from extracellular collagen fibers in the concentrated fatty tissue extract; and

d. centrifuging the treated cell preparation at a suitable speed and duration to cause the cell preparation to form: (i) a concentrated layer or pellet of stromal precursor cells, and (ii) a liquefied supernatant; and,

e. separating the layer or pellet of stromal precursor cells from the liquefied supernatant.

### EXAMPLES

### Example 1

### Extraction of Fatty Tissue via Liposuction

The patient will be sterilely prepped and draped. A skin wheal, typically on one side of the abdomen or in a thigh or buttocks area, will be raised, initially using a small and thin needle, such as a 27 gauge (27G) needle, which can deliver a saline solution containing an anesthetic such as xylocaine if desired, or which can be used after a topical anesthetic (such as a benzocaine ointment) has been applied to the skin in that area. After the initial wheal is raised using a very small needle, a larger needle (such as a 3″ 25G needle) can be used, with a fanning-style injection technique, to inject 5 cc of 1% xylocaine through the subcutaneous fat. Once this has been done, an even larger needle, such as an 18G needle, can be used if desired. When the final sharp-tipped needle is being withdrawn from the anesthetized wheal, its sharp beveled tip is used to make a somewhat enlarged nick in the skin, to accommodate an injector cannula.

A saline/xylocaine mixture is prepared using 1000 cc of 0.9% sodium chloride solution and 50 cc of xylocaine 1% with epinephrine. Of that mixture, 20 cc will be spread through the wheal area, using a rigid injector cannula coupled to a syringe. These injector cannulae are often called “Tulip injectors”, because standard and preferred models are sold by a company called Tulip Products; their website, www.tulipmedical.com, describes and illustrates devices and accessories that are commonly used during liposuction procedures.

A fanning-style injection technique should generally be used to distribute the liquids under the skin, into subcutaneous fat. The cannula should be kept generally tangential to the skin surface, so that it penetrates only shallowly into the fatty layer and does not penetrate the underlying muscles or membranes. During the injection process, lateral motion of the Tulip injector tip (which releases fluid out of the cannula via orifices on the side of the tube, rather than directly from the tip of the tube) is used to break up the fat, as fluid is being injected into and passed through the area. Generally, a semi-circular area (rather than a completely round circle) is used for extraction; however, the physician can extract fluid from an area having any size and shape, depending on factors such as the surface contours of the patient's body or limb in that region.

It should also be noted that, if desired, stromal precursor cells for use in connective tissue repair as described herein can be obtained by means of a larger-volume liposuction procedure that will also serve a weight-reduction purpose. For example, if an overweight person is having knee, hip, or ankle problems (as is fairly common), then a portion of the liquefied fatty tissue which is removed during a larger-scale liposuction procedure (using general anesthesia, a relatively large extraction cannula, etc.) can be processed as described herein, to isolate and concentrate a set of stromal precursor cells which can then be injected back into one or more joints or other areas of discomfort or damage, such as into either or both of the knee or hip joints.

After a fluid injection stage has been completed, the injector cannula will be withdrawn and replaced by a Tulip extractor. If desired, an extractor cannula can be connected to a machine which can exert either: (i) a steady suction force on the extractor tube; or, (ii) a variable level of suction, which can be controlled by the surgeon (or an assistant) by various means, such as a foot pedal. However, most surgeons prefer to have manual control over the level of suction, during at least part of the procedure, so that they can feel (with their hands) what is happening during the fluid withdrawal. Accordingly, that type of suction force is created when the surgeon pulls on the handle of the plunger, which travels inside the barrel of the syringe. If desired, the surgeon can also use a “Johnnie Lok” (illustrated at www.tulipmedical.com), which is a specialized type of clip that will temporarily affix the plunger handle to the syringe barrel, in a manner which sustains a level of suction inside the syringe while the surgeon's hands are temporarily freed up to do something else.

The liquefied fatty tissue will be extracted using a fanning technique. Each time a syringe barrel (20 cc is a preferred and convenient size) approaches a point of being full, it can be detached from the Tulip extractor, moved out of the way, and replaced by an empty 20 cc syringe. The full syringe can be either set aside for a few minutes while it awaits processing, or a surgeon's assistant can immediately begin processing the fluidized fatty tissue inside the syringe.

This extracting process can be repeated until the desired amount of fat has been harvested, using any number of 20 cc syringes.

### Example 2

### First Centrifugation Step

When a desired number of 20 cc syringes have been filled with a fluidized liposuction extract, they are placed into two holding cartridges which are designed for use in the type of centrifuge machine the surgeon is using. As described above, a preferred approach involves screwing a small “blind” cap onto the threaded luerlock tip of each syringe, before the syringe is placed in a centrifuge cartridge.

The syringe plungers, which were used to establish suction during the liposuction process, are disengaged from the syringe barrels, before the loaded syringes are centrifuged, by unscrewing the tips of the plungers from accommodating threads in plunger tip or stopper components, made from rubber or a flexible polymer. Each rubber or polymer stopper will remain in position, within a syringe barrel, during centrifugation, and will act as a watertight cap on the liquid in the syringe, which will help maintain sterility of the contents inside the syringe.

After two cartridges have been loaded with the desired number of syringes, they preferably should be weighed or balanced against each other, to ensure that they have approximately balanced weights. Whenever appropriate, additional weight should be added to a cartridge which weighs substantially less than the cartridge it is paired against.

Once the syringes have been emplaced in the cartridge, and after the plunger handles have been removed, the cartridge is placed in the centrifuge, and is centrifuged at about 40 G, for 8 to 10 minutes.

When the centrifugation of the liposuction extract is complete, there will be three main layers, which are relatively easy to distinguish, visually. In the “bottom” of the tube will be an aqueous layer, with a substantial number of stromal stem cells; accordingly, that watery fluid will be passed through a cell-retaining filter, to keep the cells in the liquid that will be retained and used, while the water is removed. To dislodge the cells from the surface of the filter, a “pulse” of a watery liquid (such as Hanks balanced salt solution, or HBSS) will be forced through the filter.

In the “top” of the tube will be an oily liquid, which will have few if any viable cells. That layer will be discarded.

The center layer, referred to herein as a “spun fat” material will contain the majority of the viable stromal stem cells. It will be saved for subsequent processing and use.

In a number of early tests, the “spun fat” layer was mixed directly with platelet-rich plasma (PRP), using the same 2:1 ratio described below, and injected back into the patient, into a connective tissue repair site. The results of most of those tests were entirely acceptable, and those treatments provided substantial and even major benefits to most of the patients who were treated with stromal stem cells from a “spun fat” layer after a single centrifugation step.

However, additional processing steps were subsequently developed, to further purify and concentrate stromal stem cells from a liposuction extract. Those additional steps are now regarded as establishing the preferred “best practice” mode for treating a liposuction extract to concentrate stromal stem cells from the extract, before the stromal stem cells are reinjected back into a patient. Accordingly, those additional processing steps are described in the next example.

### Example 3

### Collagenase Treatment and Second Centrifugation

The layer of “spun fat” from the first centrifugation step will contain a substantial amount of extra-cellular debris, including glycogen particles, and strands of collagen, the fibrous protein which creates the extra-cellular matrix that holds cells together in any type of soft tissue.

As described above, one method for breaking apart and removing the remnants of the extra-cellular collagen matrix (which otherwise can cause viable stem cells in the spun fat layer to clump together in undesirable ways) involves treating the “spun fat” from the first centrifugation step (supplemented by cells that were filtered out of the watery layer that was formed during the first centrifugation), with an aqueous salt solution, and with collagenase, an enzyme that will digest and break apart collagen fibers.

Because of the volumes that are involved, this incubation step can be carried out in a different incubation chamber, which should be designed for placement directly into a centrifuge when the collagenase incubation step is complete. Typically, a commercially-available dry bovine collagenase A type 1 preparation (sold by Sigma-Aldrich) is mixed with 25 cc of Hank's balanced salt solution (HBSS, sold by Gibco-BRL, and which should not contain phenol red indicator) to prepare a 0.2% (by weight) concentration of the collagenase. 50 cc of “spun fat” with stromal stem cells is added to the 25 cc collagenase solution, and the chamber is shaken vigorously for 5 to 10 seconds. The mixture is then incubated for an hour at 37° C., with additional shaking every 10 to 15 minutes.

At the end of that incubation period, the 75 cc cell-and-collagenase mixture is diluted 9:1 (i.e., to a final 10% level), by adding 675 cc of either or both of the following: (i) commercially-available fetal bovine serum, and/or (ii) platelet-poor plasma, preferably from the same patient who is being treated (about 25 cc of platelet-poor plasma will be generated from a 60 cc aliquot of whole blood).

The resulting mixture is vigorously shaken for several seconds, to ensure that any stromal stem cells in the collagenase-treated cell preparation can be released from any remaining collagen fragments or other debris. It is then centrifuged at about 40 G for about 8 to 10 minutes. The relatively dense stromal stem cells will settle into the bottom of the centrifuge chamber. If the bottom of the chamber is flat, the stromal stem cells will form a layer; if the bottom of the chamber is conical, the stromal stem cells will form a pellet. The supernatant is discarded, and the concentrated stromal stem cells, which will be in a relatively thick and viscous material, comparable to a paste, are ready to be mixed with platelet-rich plasma (PRP).

### Example 4

### Preparation of Platelet-Rich Plasma

To prepare a sufficient quantity of platelet-rich plasma (PRP), approximately 60 cc of blood is withdrawn from the patient. The blood can be processed directly in a SMARTPReP™ unit (sold by Harvest Technologies), which is a specialized unit that will create about 5 to 10 cc of PRP from 60 cc of whole blood. The blood should be processed promptly after withdrawal, to isolate a platelet-rich plasma extract which will be injected back into the patient; if desired, a platelet-poor plasma liquid can also be collected, and can be used during the second centrifugation step, as described above. Any platelet-rich or platelet-poor plasma preparations should be stored in a medical-grade freezer, at a suitable temperature, such as −80° C.

It should also be noted that 25 cc of platelet-poor plasma (from 60 cc of whole blood) can be passed through a “mini-heme” concentrator, which will remove most of the water, to create about 1 to 2 cc of an enriched fluid that will contain various growth factors and other polypeptides and proteins. If desired, this concentrated proteinaceous fluid can be added to the PRP liquid, before the PRP is mixed with the stem cell preparation.

The PRP preparation will be mixed with the stromal precursor cell preparation, and the mixture will be injected (with real-time imagery as a guide, such as via ultrasonic imaging) into a site in a patient's body or limb which is in need of connective tissue repair.

Thus, there has been shown and described a set of interconnected devices, machines, and methods for: (1) using minimally-invasive liposuction methods to extract stromal precursor cells from a patient who needs connective tissue repair; (2) processing the liposuction extract to create a concentrated preparation of stromal precursor cells; and, (3) reinjecting the stromal precursor cells back into the patient, along with platelet-rich plasma, at a site where connective tissue is damaged or defective.

Although this invention has been exemplified for purposes of illustration and description by reference to certain specific embodiments, it will be apparent to those skilled in the art that various modifications, alterations, and equivalents of the illustrated examples are possible. Any such changes which derive directly from the teachings herein, and which do not depart from the spirit and scope of the invention, are deemed to be covered by this invention.

1. A cartridge for holding syringes in a centrifuge cup during centrifugation, wherein said cartridge:
a. has external dimensions which allow it to be placed within and held securely by a centrifuge cup in a desktop centrifuge machine;
b. has at least two wells, each of which is capable of holding and supporting a disposable syringe that contains 20 cubic centimeters of fluid obtained from a patient via liposuction;
c. has sufficient strength and reinforcement to enable repeated use of said cartridge for centrifugal processing of syringes containing fluid obtained from patients via liposuction, at rotational speeds which generate centrifugal forces that are at least 40 times the force of gravity.

2. The cartridge of claim 1, having external dimensions which allow it to be placed within a centrifuge cup in a centrifuge machine designed and suited for preparing platelet-rich plasma from blood.

3. The cartridge of claim 1 which contains three wells, each of which is capable of holding and supporting a disposable syringe that contains 20 cubic centimeters of fluid.

4. The cartridge of claim 1 which contains three wells, each of which is capable of holding and supporting a disposable syringe which can contain 20 cubic centimeters of fluid and which has a syringe barrel with an internal diameter greater than 2 centimeters.

4. A system for centrifuging and filtering cells from body fluids, comprising:
a. a centrifuge machine of a type found in physicians' offices, which is designed and suited for preparing platelet-rich plasma from blood, and which has two centrifuge cups at opposed ends of a rotor, wherein each centrifuge cup is designed and suited for holding and accommodating a removable cartridge during centrifugation of a body fluid;
b. a plurality of centrifugation cartridges, each of which is sized and designed to fit within a centrifuge cup in said centrifuge machine, wherein each centrifugation cartridge contains at least two wells, with each well being sized and designed to hold and support a disposable syringe that can hold 20 cubic centimeters of fluid; and,
c. at least one filtration device containing fluid handling components and a filter that allows aqueous liquids to pass through said filter while retaining eukaryotic cells on at least one surface of the filter.

5. A method for treating connective tissue defects, comprising the following steps:
a. removing a liquefied fatty tissue extract containing stromal precursor cells, from a patient with a connective tissue defect;
b. centrifuging the liquefied fatty tissue extract at a speed and for a duration which separates the liquefied fatty tissue extract into an aqueous layer, a layer of concentrated fatty tissue extract containing viable cells, and an oily layer;
c. treating the concentrated fatty tissue extract by means of either mechanical processing or collagenase digestion, in a manner which to causes viable cells to be released from extracellular collagen fibers in the concentrated fatty tissue extract;
d. centrifuging the digestion mixture at a suitable speed and duration to create a concentrated preparation of stromal precursor cells;
e. injecting the concentrated preparation of stromal precursor cells, combined with platelet-rich plasma from the same patient, into a site in the patient's body that is in need of connective tissue repair.

6. The method of claim 5 wherein the aqueous layer from the liquefied fatty tissue extract is passed through a cell filter which retains cells while allowing aqueous liquid to pass through for removal and disposal, and wherein said cells which were filtered from the aqueous layer are added to the digestion mixture containing collagenase and concentrated fatty tissue extract.

7. The method of claim 5 wherein the layer of concentrated fatty tissue extract containing viable cells is treated, to cause viable cells to be released from extracellular collagen fibers in the concentrated fatty tissue extract, by means which include (i) mixing the concentrated fatty tissue extract with collagenase and an aqueous diluent to create a digestion mixture, and (ii) incubating the digestion mixture for a sufficient time to allow the collagenase to degrade extracellular collagen fibers in the fatty tissue extract;

7. The method of claim 5 wherein the layer of concentrated fatty tissue extract containing viable cells is treated, to cause viable cells to be released from extracellular collagen fibers in the concentrated fatty tissue extract, by means which include mechanical processing which generates shearing forces that will cause viable cells to be released from extracellular collagen fibers.

8. The method of claim 5 wherein the mechanical processing which generates shearing forces that will cause viable cells to be released from extracellular collagen fibers is selected from the group consisting of pressurized extrusion, screen passaging, and shearing-force stirring.

9. A cell preparation for treating connective tissue defects, created by steps comprising the following:
a. removing a liquefied fatty tissue extract containing stromal precursor cells, from a patient with a connective tissue defect;
b. centrifuging the liquefied fatty tissue extract at a speed and for a duration which separates the liquefied fatty tissue extract into an aqueous layer, a layer of concentrated fatty tissue extract containing viable cells, and an oily layer;
c. mixing the concentrated fatty tissue extract with collagenase and an aqueous diluent to create a digestion mixture;
d. incubating the digestion mixture for a sufficient time to allow the collagenase to degrade extracellular collagen fibers in the fatty tissue extract; and
e. centrifuging the digestion mixture at a suitable speed and duration to cause the digestion mixture to form: (i) a concentrated layer or pellet of stromal precursor cells, and (ii) a liquefied supernatant; and,
f. removing at least a substantial portion of the supernatant, from the layer or pellet of cells.

10. A cell preparation for treating connective tissue defects, created by steps comprising the following:
a. removing a liquefied fatty tissue extract containing stromal precursor cells, from a patient with a connective tissue defect;
b. centrifuging the liquefied fatty tissue extract at a speed and for a duration which separates the liquefied fatty tissue extract into an aqueous layer, a layer of concentrated fatty tissue extract containing viable cells, and an oily layer;
c. subjecting the concentrated fatty tissue extract to mechanical processing which generates shearing forces that will cause viable cells to be released from extracellular collagen fibers, thereby creating a fluidized suspension containing detached stromal precursor cells, and extracellular debris;
e. centrifuging the fluidized suspension containing detached stromal precursor cells and extracellular debris at a suitable speed and duration to cause the digestion mixture to form: (i) a concentrated layer or pellet of stromal precursor cells, and (ii) a supernatant; and,
f. removing at least a substantial portion of the supernatant, from the layer or pellet of cells.
